(19) United States
(12) Patent Application Publication
(10) Pub. No.: US 2006/0224095 A1
(43) Pub. Date: Oct. 5, 2006

(54) Biocompatible polymeric vesicles self assembled from triblock copolymers

(75) Inventors: Jerome Claverie, Durham, NH (US); Floraine Collette, Kittery, ME (US)

Correspondence Address: Kevin M. Farrell;Pierce Atwood, Suite 350, One New Hampshire Avenue, Portsmouth, NH, 03801, US

(73) Assignee: University of New Hampshire, Durham, NH (US)

(21) Appl. No.: 11/098,843

(22) Filed: Apr. 5, 2005

(51) Int. Cl. A61F 5/00

(52) U.S. Cl. 602/5

(57) ABSTRACT

Provided herein is a novel composition useful in assembling carrier vesicles for delivery of a biologically active agent to an animal. The composition comprises an amphiphilic triblock copolymer comprised entirely of biocompatible, biodegradable, and/or enzymatically degradable polymers. The composition is characterized by the ability to self assemble into an aqueous vesicle, thereby encapsulating an agent for delivery to the animal. Also provided is a method for making a composition for delivery of an agent and a method for administering the agent to an animal.

### BACKGROUND OF THE INVENTION

The delivery of pharmaceutic active principles or agents, and among those, of therapeutic proteins and other macromolecules, can present significant challenges. Most if not all therapeutic proteins are delivered parenterally. Using less invasive delivery methods would be highly beneficial, since it would bypass the use of painful injections, which have a high risk of immunological cross reactivity.

It has also long been acknowledged that vehicles able to deliver internally active pharmaceutical agents must be of relatively small size, since, without exception, the animal body is impermeable to any large sized objects. In general, the transition from small to large occurs at several thousands of nanometers, and thus nanotechnology is aptly suited for the preparation of pharmaceutical delivery vehicles. Small size is a necessary but not sufficient requirement for a successful delivery vehicle. Additionally, the delivery vehicle must be constituted of biocompatible components and should not interfere with the active principle or agent. For example, nanoparticles and polymeric micelles bearing hydrophobic cores have been determined to be unsuitable for delivery of therapeutic proteins since such proteins typically unfold in an encapsulated hydrophobic environment and thereby lose activity.

Vesicles are containers that enclose a volume with a very thin membrane. Liposomes are vesicles that have been widely used for encapsulating active pharmaceutical components. Liposomes are formed upon the self-assembly of phospholipids in a continuous bilayer.

It is frequently desirable to shield the active ingredient incorporated in the vesicle from the external environment, since the external environment can contain enzymes that have the capacity to degrade the active components. As such, a stable membrane is an important component of a pharmaceutical vehicle. In the art, a vesicle typically ranges from about 10 nm to about 10,000 nm in diameter, with a membrane width usually less than about 5 nm for liposomes. Practical applications of liposomes have been hindered by a lack of stability and uncontrolled leakage of the encapsulated compound from the vesicle (Lasic, D. D. and D. Papahadjopoulos (1998) “Medical Applications of Liposomes” New York, Elsevier), problems presumably arising from the lack of stability attributed to the vesicle from the small dimension of the membrane.

Vesicles with more stable membranes have been prepared by self assembly of controlled polymeric systems. Vesicles have been prepared by self assembly of amphiphilic triblock copolymers A-B-C, where A and C are water soluble and B is oil-soluble. A and C can have different or similar chemical nature. The triblock poly(2-methyloxazoline)-block-poly(dimethylsiloxane)-block-poly(2-methyloxazoline) has been shown to spontaneously form vesicles in water as shown in “Nardin, C., T. Hirt, et al. (2000) Polymerized ABA triblock copolymer vesicles Langmuir 16: 1035” and in “Nardin, C., S. Thoeni, et al. (2000) Nanoreactors based on Polymerized ABA-triblock copolymer vesicles, Chem. Comm, 1433”. Vesicles have also been formed by the self assembly of poly(ethylene oxide)-block-poly(propylene oxide)-block-poly(ethylene oxide) as described in “Schillen, K., K. Bryskhe, et al. (1999). Vesicles formed from PEO-PPO-PEO triblock copolymer in dilute aqueous solution Macromolecules 32: 6885-6888”. The described vesicles however are not ideal for delivery of an agent to an animal since the A and B blocks in these vesicles are not biodegradable and in the case of PEO-PPO-PEO are not sufficiently stable as the vesicular solution reverts to a lamellar phase over time.

In WO 2004/009664 entitled “Biodegradable triblock copolymers, synthesis methods thereof, and hydrogels and biomaterials made therefrom”, the use of poly(ethylene oxide)-block-poly(hydroxybutyrate)-block-poly(ethylene oxide) is described for drug delivery applications. The B block of the described polymer is biodegradable, and the A and C blocks are biocompatible, but the A block is not enzymatically degradable, a feature desirable to facilitate release of an encapsulated agent inside the body of an animal. If the active principle or agent encapsulated in a vesicle is completely shielded from an external medium, it is then inefficient as a drug because it does not interact directly with the body. This reference also describes methods to form a hydrogel from a triblock copolymer in the presence of a cyclodextrin, but the disclosed triblock copolymers have not been demonstrated to form vesicles in water. Thus, there is a need for stable biocompatible nanocapsules with hydrophilic, biodegradable, and enzymatically degradable components and drug delivery applications for use of the same.

### SUMMARY OF THE INVENTION

The invention, in one aspect, is directed to polymeric compositions useful in the preparation of nanoscale vehicles for drug delivery. This composition includes a novel non-phospholipid containing amphiphilic triblock ABC copolymer characterized by the ability to self assemble into an aqueous vesicle. The A block is characterized as biocompatible, hydrophilic, and enzymatically degradable. The B block is characterized as biocompatible, biodegradable and hydrophobic. The C block is characterized as biocompatible and hydrophilic. Individual polymer blocks may be composed of a homogenous or heterogenous mixture of monomers or oligomers. The A and C blocks may comprise the same polymer or may alternatively comprise different polymers. The lengths and hence size of each of the A, B, and C polymer blocks can vary, and depend ultimately on the desired size of the aqueous vesicle to be generated by such polymer blocks. The triblock copolymer preferably contains a number of polymer molecules to form an aqueous vesicle with an average diameter of about 5 nm to about 10,000 nm, or more preferably in the range of about 50 nm to about 200 nm.

The invention is also directed to an aqueous vesicle comprising an amphiphilic triblock ABC copolymer of the present invention and an agent encapsulated in the aqueous vesicle. In self assembling into aqueous vesicles, the individual triblock copolymer molecules form closed polymer shells generally spherical in nature. The closed polymer shells shield an encapsulated agent for delivery from conditions which might degrade or inactivate the agent in the body of an animal. An aqueous vesicle of the present invention may include other components which do not interfere with its ability to self assemble into a vesicle and do not alter its biocompatible and/or biodegradable properties. Size distribution of assembled vesicles may be controlled by methods known in the art, with desired size depending ultimately on the tissue to which delivery is targeted. The aqueous vesicle allows for delivery of biologically active agents which would otherwise be degraded prior to sorption by the body. Suitable agents include proteins, polypeptides, peptides, nucleic acids, and synthetic organic molecules, or a mimetic of any one of the same. Nucleic acids may be single-stranded or double-stranded DNA or RNA molecules and may further include oligonucleotides, plasmids, and vectors. The agent may be a therapeutic, prophylactic, diagnostic, or other agent.

The invention is also directed to a method for making a vesicle composition for delivery of an agent. This method includes contacting the non-phospholipid containing amphiphilic triblock copolymer with an aqueous solution containing an agent to be delivered. Contact with the aqueous solution is effective to prompt self assembly of the non-phospholipid containing amphiphilic triblock copolymer into an aqueous vesicle and thereby encapsulate the agent for delivery.

Also provided is a method for using a composition of the present invention for administering an agent to a non-human or human animal. Self assembly of the aqueous vesicle with an encapsulated agent may occur either inside or outside the body of an animal. Aqueous vesicles assembled from non-phospholipid containing amphiphilic triblock copolymers outside the body may be delivered lyophilized or in aqueous form. Aqueous vesicles assembled from non-phospholipid containing amphiphilic triblock copolymers inside the body may be delivered as a formulation of an agent with the copolymer, and the vesicle encapsulating the agent formed one the formulation is exposed to the hydrophilic environment inside the body of the animal. The agent to be delivered may be for prophylactic, diagnostic, therapeutic, or other purpose.

### DESCRIPTION OF THE DRAWINGS

FIG. 1 shows the vesicle size distribution obtained by dynamic light scattering after self assembly, following extrusion through a 0.45 μm filter, and following extrusion through a 0.22 μm filter.

FIG. 2 shows the degradation of a suspension of vesicles by an enzyme (protease, Type I, from bovine pancreas) at different times (indicated in minutes). The left vial is a vial containing vesicles and no enzyme. The right vial, labeled P, contains the vesicles and the enzymes.

FIG. 3 shows the average insulin level in the blood of rats for each group, indicating that the polymer was effective in promoting the oral delivery of insulin. Group 1: insulin solution (0.04 units) injected subcutaneously. Group 2: insulin solution (20 units) fed by oral gavage. Group 3: insulin solution (20 units) and polymer 2.1 fed by oral gavage. Group 4: insulin (4 units) and polymer 2.1 in a gastroresistant formulation fed by oral gavage.

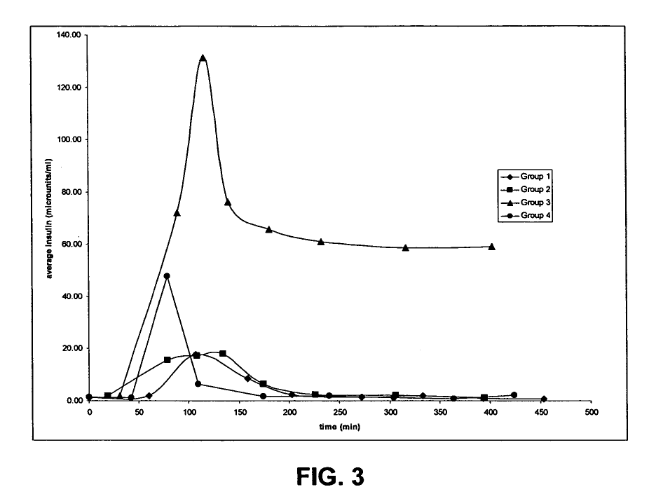

### DETAILED DESCRIPTION OF THE INVENTION

The present invention is based on the discovery of a new amphiphilic triblock copolymer useful for drug delivery applications. An amphiphilic triblock copolymer of the present invention is characterized by the ability to self assemble into an aqueous vesicle and encapsulate an agent for delivery of the agent to an animal. The triblock copolymer represents the first example of a composition comprised entirely of biocompatible and/or biodegradable blocks able to self assemble into an aqueous vesicle. Provided herein is a composition comprising the triblock copolymer, the aqueous vesicle containing this composition, and methods for making and using the ABC triblock copolymer-containing vesicle.

Copolymer Composition

In one aspect the present invention relates to a composition comprising a non-phospholipid containing amphiphilic triblock ABC copolymer. The identity of the A, B, and C polymer blocks is restricted only by the properties the individual polymer blocks impose on the copolymer. The A block of the copolymer is characterized as biocompatible, hydrophilic, and enzymatically degradable. The B polymer block is characterized as biocompatible, biodegradable and hydrophobic. The C polymer block is characterized as biocompatible and hydrophilic. A triblock copolymer comprising an A polymer block that is enzymatically degradable and biocompatible, B and C polymer blocks that are biocompatible, and a B polymer block that is biodegradable allows its use upon assembly into an aqueous vesicle as a vehicle for delivery of an agent to an animal such as a human. A triblock copolymer comprising an A polymer block that is enzymatically degradable and biocompatible confers the triblock-containing aqueous vesicle the ability to release an encapsulated agent in a controlled manner, such as upon contact with enzymes that are inside or outside a cell.

Individual polymer blocks may be composed of a homogenous or heterogenous mixture of monomers or oligomers. The copolymer is not to be restricted by the properties of the individual monomers or oligomers, but rather by the properties imparted by the monomers and/or oligomers to the polymer blocks as a whole. Preferably, the hydrophobic B block contains a predominant amount of hydrophobic monomeric units, and preferably, the hydrophilic A and C blocks contain a predominant amount of hydrophilic monomeric units. Specific examples of monomeric units which may comprise the A polymer block and impart biocompatible, hydrophilic, and enzymatically degradable properties include glutamic acid, aspartic acid, lysine, serine, asparagine, histidine, tyrosine, allose, altrose, glucose, mannose, gulose, idose, galactose, talose, ribose, arabinose, xylose, lyxose, ribulose, xylulose, psicose, fructose, sorbose and tagatose. The A polymer block may comprise polyglutamic acid, polyaspartic acid, polylysine, polyserine, polyasparagine, polyhistidine, polytyrosine and water soluble polysaccharides and carbohydrates. Specific examples of monomeric units which may comprise the B polymer block and impart biodegradable and hydrophobic properties include lactic acid, glycolic acid, epsilon-caprolactone, trimethylene carbonate, p-dioxanone, morpholine-2,5-dione, glycosalicylate, 3,3-dimethyltrimethylene carbonate, 1,4-dioxapane-2-one, sebacic acid and adipic acid. Compounds suitable as B polymer block compounds include polylactide, polyglycolide, poly-epsilon-caprolactone, poly[trimethylene carbonate, p-dioxanone], poly[morpholine-2,5-dione], poly[glycosalicylate], poly[3,3-dimethyltrimethylene carbonate], poly[1,4-dioxapane-2-one] and polyesters and polyanhydrides derived from sebacic and adipic acid. Specific examples of monomeric units which may comprise the C polymer block and impart biocompatible and hydrophilic properties include ethylene glycol, glutamic acid, aspartic acid, lysine, serine, asparagine, histidine, tyrosine, allose, altrose, glucose, mannose, gulose, idose, galactose, talose, ribose, arabinose, xylose, lyxose, ribulose, xylulose, psicose, fructose, sorbose and tagatose. Specific examples of compounds suitable as C polymer block compounds include polyethylene glycol, polyglutamic acid, polyaspartic acid, polylysine, polyserine, polyasparagine, polyhistidine, polytyrosine and water soluble polysaccharides and carbohydrates. A triblock ABC copolymer of the present invention may further comprise Poly(glutamic acid-b-lactide-b-ethylene glycol).

In the context of the present invention the term “block” or “polymer block” refers to a segment of the copolymer. The segment can be linear, branched or hyperbranched. It can be composed of one or several monomeric units. Also in the context of the present invention, the term “biocompatible” is intended to mean that the stated polymer composition does not produce a toxic, injurious or immunological response upon delivery to an animal. The terms “hydrophilic” and “hydrophobic” are intended to mean that the stated polymer compositions are soluble and insoluble, respectively, in water or in a buffer at the concentration of usage. The term “biodegradable” is intended to mean that a stated polymer composition is capable of being broken down or degraded, generally by hydrolysis or enzymatic digestion, within an animal to which the composition has been delivered. Although there does not exist a complete list of biocompatible or biodegradable polymers which exist up to date, a wide range of these materials can be found in the section of the Aldrich® catalog “Products for Materials Science” called “Biocompatible/Biodegradable Polymers”. Biocompatible polymers are most often constituted of repeat units which are non-toxic and naturally found in the body. Biodegradable polymers most often contain ester, amide, anhydride, ketal, carbonate and urea groups in the repeat unit along the main chain. For example, polyethylene glycol, polyethylene or polybutadiene, which do not contain any ester, amide, anhydride, ketal, carbonate and urea groups, are not biodegradable. Polyethylene glycol (number average molecular weight<10,000 g/mol) is biocompatible, whereas polyethylene and polybutadiene are not when incorporated into drug delivery carriers. Although polyethylene glycol (number average molecular weight<10,000 g/mol) is not biodegradable, it is generally regarded as safe, since it is excreted in urine (see for example Brady C E, DiPalma J A, Morawski S G, Santa Ana Calif., Fordtran J S, Gastroenterology. 1986 June;90(6):1914-8, Mehvar, R. J. Pharm. Pharmaceut. Sci., 3(1):125-136, 2000). The term “enzymatically degradable” is intended to mean that the stated composition is capable of being cleaved or digested, either partially or extensively, by enzymes. For example, polyamino acids, proteins, polysaccharides, carbohydrates and nucleotides are usually enzymatically degradable. The term “encapsulate” is intended to refer to the formation of physical barrier between the hollow inner shell of a vesicle and the environment outside the vesicle. The barrier is intended to be impermeable to macromolecules and water soluble organic molecules in the absence of any biodegradative or enzymatic action on the triblock copolymer.

The A and C blocks may comprise the same polymer or may alternatively comprise different polymers. Triblock copolymers wherein the A and C polymer blocks are comprised of the same material are typically referred to in the art as ABA triblock copolymers. As such, ABA triblock copolymers which meet the criteria stated herein fall within the scope of the present invention but will be referred to as ABC copolymers for simplicity. Examples of polymers suitable for inclusion as A and/or C block polymers include polyglutamic acid, polyaspartic acid, polylysine, polyserine, polyasparagine, polyhistidine, polytyrosine and water soluble polysaccharides and carbohydrates.

The lengths and hence size of each of the A, B, and C polymer blocks can vary, and depend ultimately upon the desired size of aqueous vesicle to be generated by such polymer blocks. The lengths of the individual hydrophobic and hydrophilic polymer blocks can be controlled in part by increasing or decreasing concentrations of starting materials in the polymerization reactions. The lengths of the individual hydrophobic and hydrophilic polymer blocks can also be influenced by controlled reaction conditions such as temperature for the polymerization. Each block is obtained by a controlled or a living polymerization process which is known to the skilled researcher to yield polymers of low polydispersities. Over the vast choice of polymerization methods available, anionic ring-opening polymerization, pseudo-living cationic polymerization and coordinated ring-opening polymerization will be preferred over step-growth polymerization, radical polymerization, conventional cationic polymerization, anionic polymerization, catalytic polymerization and conventional ring-opening polymerization as the latter methods usually yield polydisperse polymers or non-biocompatible polymers. It is important, but not necessary for the embodiments of the present invention, to prepare polymers which have the lowest possible polydispersity. The molecular weight of each block affects the nature of the self-assembled object. For an ABC triblock copolymer with polydisperse blocks, the self assembly step may become ill-defined and irreproducible. The use of controlled or living polymerization methods such as those encountered in anionic ring-opening polymerization, pseudo living cationic polymerization and coordinated ring-opening polymerization are also methods of choice for the preparation of triblock copolymers ABC, since the synthesis of block copolymers is greatly facilitated by the control/living characteristics of the polymerization. Biocompatible and monodisperse polymers can also be prepared by other methods such as bacterial production and solid or liquid phase sequential synthesis. Typically, each of the A, B, and C polymer blocks is characterized by a number average molecular weight of at least 500 g/mol. Preferably, the A polymer block is characterized by a number average molecular weight in the range of 1,000 g/mol-50,000 g/mol. Preferably, the B polymer block is characterized by a number average molecular weight in the range of 1,000 g/mol-30,000 g/mol, or more preferably in the range of 2,000 g/mol-15,000 g/mol. Preferably, the C polymer block is characterized by a number average molecular weight in the range of 1,000 g/mol-10,000 g/mol.

Aqueous Vesicle Compositions Comprising the ABC Triblock Copolymer

The copolymer is characterized by its ability to self assemble into an aqueous vesicle, and it is an object of the invention to provide for an aqueous vesicle comprising an amphiphilic triblock ABC copolymer of the present invention. Self assembly occurs in the presence of a solvent and, although not required, may occur in the presence of water or other aqueous containing solution. Self assembly of aqueous vesicles in the presence of a non-toxic medium such as water may be preferred wherein the vesicles are to be used for therapeutic delivery of an agent to an animal. Self assembly may occur either inside or outside the body of an animal. Aqueous vesicles assembled outside the body may subsequently be lyophilized and delivered to an animal in such form, and the aqueous vesicles spontaneously reformed once its components are exposed to the hydrophilic environment inside the body of an animal. Lyophilized vesicles may also be resuspended in solvent for re-assembly prior to administration to an animal. Alternatively, a formulation of an agent may be administered to an animal in the presence of the ABC triblocks of the present invention, and an aqueous vesicle encapsulating such agent be formed once the formulation is exposed to the hydrophilic environment inside the body of an animal. Self-assembly techniques may be employed as described in the art (see, for example, I. Astafieva, K. Khougaz, A. Eisenberg, Macromolecules, 1995, 28, 7127-7134, G. Yu, A. Eisenberg, Macromolecules, 1998, 31, 5546-5549, H. Shen, A. Eisenberg, Macromolecules, 2000, 2561-2572).

An aqueous vesicle of the invention may include other components which do not interfere with its ability to self assemble into a vesicle and do not alter its biocompatible, and/or biodegradable properties. Such components may be included to enhance some property of the vesicle such as its size, permeation properties, hydrophobicity, hydrophilicity, and/or charge or alternatively to enable delivery of the vesicle to a specific desired target within the animal. As an example, the surface of the vesicle may be modified by the addition of ligands specific for receptors of a cell or tissue type to which delivery of the agent is desired. As an example, antibodies for a cancer antigen so attached may be used to direct the vesicles to a cancer cell expressing the antigen. Other non-limiting examples of ligands suitable for targeting vesicles to specific cell types include carbohydrates, proteins, folic acid, peptides, permeation enhancers and peptoids. Comonomers may be added during polymerization of the polymer blocks. Inclusion of comonomers and targeting ligands is described in U.S. Pat. No. 6,616,946, the contents of which are herein incorporated by reference. Biocompatible polymers may be added to the ABC triblock upon self assembly of the polymer blocks into aqueous vesicles.

In self assembling into aqueous vesicles, the individual triblock copolymer molecules form closed polymer shells generally spherical in nature. The closed polymer shells shield an encapsulated agent for delivery from conditions which might degrade or inactivate the agent if delivered in the absence of the vesicle. As an example, an aqueous vesicle of the present invention would allow for oral delivery of agents such as small peptides, which would otherwise likely be enzymatically degraded prior to sorption by the body.

The term “aqueous vesicle” is intended to refer to spontaneously forming nanoscale structures containing an ABC triblock copolymer, with internal and external aqueous phases. Only aqueous vesicles are considered, as organic vesicles, which are generated in toxic organic solvents, are suitable for the purpose of drug delivery. Aqueous vesicles of the invention are generally spherical in shape with an internal, hollow void. Upon self assembly, whether self assembly occurs inside or outside the body of an animal, a vesicle of the present invention is stabilized for delivery. Because of the vesicle's inherent stability, the vesicle does not require, and is preferably is not subjected to, induced crosslinking once the vesicle is formed. Rather, an aqueous vesicle of the present invention is stabilized through the strength of hydrophobic interactions between the hydrophobic segments of such copolymers and through the strong segregation between the hydrophilic and hydrophobic fragments. Additional stabilization can be gained by specific interactions such as crystallization and electrostatic interactions. The identity of the A, B, and C polymer blocks of the present invention are chosen such that the hydrophilic and hydrophobic properties of the polymer blocks impart stability sufficient to encapsulate an agent for the delivery to the desired cells within an animal.

The position of the A, B, and C polymer blocks relative to one another within an aqueous vesicle of the present invention is restricted only by the properties the position of the blocks may impose on an assembled vesicle. While not wishing to be bound by theory, the outer shell of the aqueous vesicle comprises, at least in part, the A polymer block. Also while not wishing to be bound by theory, the outer shell of the aqueous vesicle may further comprise a C polymer block. And while further not wishing to be bound by theory, the B block forms a hydrophobic membrane central layer between hydrophilic inner and outer layers comprised of A and C block polymers. Decoration of both sides of the B block with hydrophilic A and/or C components likely prevents contact of any active agent with the hydrophobic B block. Also while not wishing to be bound by theory, it is presumed that the hydrophobic membrane forms the physical barrier between the inner and outer shell of the vesicle, thereby encapsulating an agent for delivery.

The triblock copolymer preferably contains a number of polymer molecules to form an aqueous vesicle with an average diameter of about 5 nm to about 10,000 nm, or more preferably in the range of about 50 nm to about 200 nm. The average size and size distribution of an aqueous vesicle of the present invention may vary, and depends upon the molecular weight and initial concentration of each polymer block in solution prior to self assembly. Size distribution of the prepared vesicles may be controlled by methods known in the art. If an agent is to be targeted to the blood, vesicles of intermediate size may be desired (20-120 nm). If an agent is to be targeted to the brain, smaller vesicles may be desired in order to facilitate traversal of the agent across the blood-brain barrier. The desirable size of a vesicle may also be determined by the size of the agent whose encapsulation within the vesicle is desired.

One of skill in the art would expect that a small aqueous vesicle of the present invention be able to traverse the blood-brain barrier and deliver an encapsulated agent to the brain. Success in traversing the blood-brain barrier has been demonstrated in the art with agents encapsulated in liposomes. Since the vesicles of the present invention possess many attributes similar to those of liposomes (e.g., size, external charge), the vesicles of the present invention would be expected to cross the blood-brain barrier with similar success. As an example, a technology which was used with liposomes and may be adapted in the context of the present invention includes attachment of a ligand to the surface of the vesicle, as stated above, to facilitate traversal of the vesicle across the blood-brain barrier. Such a ligand acts as a transporter molecule to ferry an agent-encapsulated vesicle in what is known in the art as a “molecular Trojan Horse.” Polymer-coated liposomes have successfully been used in the art in this manner and thus would be expected to similarly work in the context of the present invention.

Regardless of the conditions of self assembly, vesicles ranging in various sizes will be obtained. Subsequent to the self assembly reaction, vesicles of a uniform desired size may be obtained by methods known in the art. For example, extrusion of a vesicle containing suspension through filters with a pore width of desired size would result in obtaining mostly unilamellar vesicles of relatively uniform distribution in size. In this manner, the average diameter of obtained vesicles is directly determined by the size of the pore width in the filter membrane.

It is an object of the invention to exclude liposomal aqueous vesicles from embodiments of the present invention, which may be considered in the art as amphiphilic triblock copolymers. Resultingly, it is a requirement that a triblock copolymer of the present invention not be comprised of phospholipids in the same manner that a liposome provides an encapsulated shell comprised of phospholipids.

Agents for Encapsulation in an Aqueous Vesicle

An aqueous vesicle of the present invention is suitable for encapsulating a wide variety of agents, including but not limited to therapeutic, prophylactic, and diagnostic agents. The molecular size of an agent is generally not limiting, as both large and small molecular weight agents may be encapsulated. If necessary, larger vesicles may be used to accommodate larger molecules as agents and smaller vesicles may be used to accommodate smaller molecules as agents. Although both generally hydrophilic and generally hydrophobic agents may be encapsulated and delivered using such vesicles, it is a requirement that an agent be at least partially soluble in water. Non-limiting examples of therapeutic agents include proteins, polypeptides, peptides, nucleic acids, and synthetic organic molecules, or a mimetic of any one of the same. A nucleic acid may be a single-stranded or double-stranded DNA or RNA molecule and may further comprise an oligonucleotide. The nucleic acid may further comprise a plasmid such as a vector. Additionally, an agent may be modified prior to encapsulation, such as by glycosylation in the case of a protein, polypeptide, or peptide, or by the incorporation of analogues or labels for a nucleic acid. Therapeutic agents may function as hormones, vaccines, antibodies, antibiotics, chemotherapeutics, antisense, antiangiogenic agents, small interfering RNAs (siRNAs), or other function. Non-limiting examples of diagnostic agents include metal particles, radiolabels, and magnetic particles.

The aqueous vesicles containing encapsulated agents may be packaged in dosage forms. Aqueous vesicles containing encapsulated agents may be packaged alone in such form or in combination with other active agents. Aqueous vesicles may further be packaged with an inert carrier that allows delivery of the vesicles as a tablet, capsule, or implant. For example, for oral delivery, the vesicle can be packaged in gastroresistant pills which would allow the vesicle to bypass the acidic environment of the stomach. The number of vesicles for a particular dose may vary, depending on the amount of agent encapsulated by the vesicle. Higher or lower dosages may be attained in such form by increasing or decreasing, respectively, the number of aqueous vesicles comprising encapsulated agents or by increasing or decreasing the amount of agent encapsulated within each vesicle during assembly. In lieu of aqueous vesicles containing encapsulated agents, a mixture of an agent and triblock copolymer of the present invention may be packaged and delivered in dosage unit forms in the same manner as stated above.

Method for Making Vesicle Compositions for Delivery of an Agent

Also provided herein is a method for making a vesicle composition for delivery of an agent. This method comprises first providing any non-phospholipid containing amphiphilic triblock ABC copolymer of the present invention, wherein the A block is characterized as biocompatible, hydrophilic, and enzymatically degradable, wherein the B block is characterized as biodegradable and hydrophobic, and wherein the C block is characterized as biocompatible and hydrophilic, and further wherein the ABC triblock copolymer is characterized by the ability to self assemble into the aqueous vesicle. The method thereafter comprises contacting the non-phospholipid containing amphiphilic triblock ABC copolymer with an aqueous solution containing the agent to be delivered, effective to form an aqueous vesicle comprising the agent encapsulated in the vesicle comprising the non-phospholipid containing amphiphilic triblock ABC copolymer, thereby forming a composition for the delivery of the agent. The aqueous solution may be pure or buffered water or other non-toxic water-containing solution. Control of size distribution may be achieved as stated above.

Method for Administering an Agent to an Animal

Also provided herein is a method for using a composition of the present invention for administering an agent to an animal. This method includes first providing a composition comprising any amphiphilic triblock ABC copolymer of the present invention and an agent whose delivery to an animal is desired. The amphiphilic triblock ABC copolymer comprises an A block characterized as biocompatible, hydrophilic, and enzymatically degradable, a B block characterized as biocompatible, biodegradable and hydrophobic, and a C block characterized as biocompatible and hydrophilic. The ABC triblock copolymer is characterized by the ability to self assemble into an aqueous vesicle. The agent is characterized by the ability to be encapsulated in the self-assembled aqueous vesicle. The composition comprising the amphiphilic triblock copolymer and agent is to be administered to an animal to which delivery of the agent is desired. The animal may be either non-human or human. The agent may be encapsulated in the self-assembled vesicle prior to or subsequent to delivery of the composition. That is, self assembly of the vesicle may occur either inside or outside the body of an animal. Aqueous vesicles assembled outside the body may be delivered in lyophilized or aqueous form. When delivered lyophilized, the aqueous vesicles spontaneously reform once the composition is exposed to the hydrophilic environment inside the body of an animal.

The aqueous vesicles containing encapsulated agents may be administered in dosage units. Aqueous vesicles containing encapsulated agents may be administered alone in such form or in combination with other active agents. Aqueous vesicles may further be administered with an inert carrier that allows delivery of the vesicles as a tablet, capsule, or implant. The number of vesicles for a particular dose may vary, depending on the amount of agent encapsulated by the vesicle. Higher or lower dosages may be attained in such form by increasing or decreasing, respectively, the number of aqueous vesicles comprising encapsulated agents or by increasing or decreasing the amount of agent encapsulated within each vesicle during assembly. In lieu of aqueous vesicles containing encapsulated agents, a mixture of an agent and triblock copolymer of the present invention may be administered in dosage unit forms in the same manner as stated above.

Delivery of an agent is not limited to any particular route, with a preferred delivery dependent upon the desired treatment. Delivery of the composition may be achieved by oral, intranasal, vaginal, peritoneal, dermal, rectal, ocular, bucal, parenteral and pulmonary routes. As an example, such vesicles could be delivered in the intestine via a gastroresistant pill or with an enteric coating. Once dispersed in the lumen, enzymes degrade the A block, exposing the B-block hydrophobic layer which may thereafter interact via hydrophobic interactions with intestinal mucus and epithelial cells. Because the hydrophobic layer is also biodegradable, especially at the pH of the mucus and of the endosome (pH˜4.5 vs 7.4 in the lumen of the intestine), the agent is eventually released in the cell or at the proximity of the cell.

Once delivered to an animal, a vesicle of the invention is biodegraded and enzymatically degraded, enabling release of an encapsulated agent within the body of the animal.

### Other Embodiments

It is to be understood that, while the invention has been described in conjunction with the detailed description, thereof, the foregoing description is intended to illustrate and not limit the scope of the invention. Other aspects, advantages, and modifications of the invention are within the scope of the claims set forth below.

### Exemplification

Experiment 1.1 Diblock Copolymer Poly(ethylene glycol-b-lactide)

| | weight | volume |
|---|---|---|
| Reagent | (g) | (mL) |
| Et₂Zn | | 0.45 |
| PEG | 2 | |
| rac-lactide | 10 | |
| toluene (dried) | | 80 |
| HCl | | 0.26 |
| THF | | 50 |
| diethyl ether | | 250 |

This experiment was realized using standard Schlenk techniques required to manipulate air-sensitive reagents. The toluene (1 L), used in this experiment, was magnetically stirred for 24 hours in the presence of calcium hydride (10 g). It was then distilled under reduced pressure, and stored under argon. Racemic lactide (rac-lactide, Aldrich) was recrystallized from the commercially available form and dried under vacuum for 24 hours. The racemic lactide was dissolved in 60 ml of toluene, and the solution was then heated at 60° C. for one hour and half. Polyethylene glycol monomethyl ether (2 g, Aldrich, Mn=2000 g/mol, viscosity 54,6000 centistokes) was added to 20 mL of dried toluene in a separate round bottom flask and was stirred until complete dissolution. Diethyl zinc (0.45 ml, Aldrich, 1.1 mol/L in toluene) was added via an argon-flushed syringe to the polyethylene glycol solution, and the mixture was stirred magnetically for one hour. Then, the zinc containing solution was transferred via a cannula to the lactide containing solution. The mixture was left to react at 60° C. for one hour. Then, 0.26 mL of hydrochloric acid (Fisher, metal grade, 35.5% in water) was added, and the solvent was subsequently evaporated using a rotary evaporator. The diblock was then redissolved in 50 ml of THF. The solution was added to a magnetically stirred beaker containing 250 ml of ether kept at −18° C. The solid was separated from the liquid by filtration over a fritted glass filter (pore size: 10 to 16 microns) and was washed twice with cold ether (2*50 mL). It was then dried under vacuum (residual pressure<100 microtorrs) for 12 hours at room temperature. 8.9 g of diblock copolymer were collected.

Analysis:

The solid was analyzed by gel permeation chromatography (GPC) and nuclear magnetic resonance of the proton (¹H NMR).

GPC

The GPC instrument was constituted of an isocratic HPLC pump Waters 515, a refractometric detector Waters 2414, an autosampler Waters 717 plus, a monowavelength UV-VIS detector Waters 486, an oven (Waters temperature control module). The acquisition and treatment software was Millenium 32. The instrument was equipped with a series of four columns from Polymer Laboratories (PLgel 5 um 100A, PLgel 3 um 100A, PLgel 5 um MIXED-C, PLgel 5 um MIXED-C). Tetrahydrofuran was used as eluent at 35° C. and the flow rate was set at 1 mL/min. The calibration was done using polystyrene standards ranging in molecular weight from 695 to 361,000 g/mol. The molecular weight (polystyrene standard) of the solid is Mₙ=12411 g/mol, M_{w}/Mₙ=1.139. The chromatogram indicates that there is no more residual polyethylene glycol left, indicating that a diblock copolymer has been obtained.

NMR

¹H NMR analysis was realized on a Varian Mercury 400 MHz NMR using CDCl₃ as solvent (relaxation delay of one second, 45 degree pulse, 16 repetitions). TMS was used as reference.

¹H NMR (400 MHz)

| solvent | | CDCl₃ (δ, |
|---|---|---|
| signal | | ppm) |
| PEG | CH₂ | 3.7 |
| PLA | CH | 5.2 |
| | CH₃ | 1.55 |

Experiment 1.2 Diblock Copolymer Poly(ethylene glycol-b-lactide)

| | weight | volume |
|---|---|---|
| Reagent | (g) | (mL) |
| Et₂Zn | | 0.35 |
| PEG | 1.54 | |
| Lactide (90% L/ | 7.69 | |
| 10% rac) | | |
| toluene (dried) | | 61 |
| HCl | | 0.2 |
| THF | | 50 |
| diethyl ether | | 250 |

This experiment was realized using standard Schlenk techniques required to manipulate air-sensitive reagents. The toluene (1 L), used in this experiment, was magnetically stirred for 24 hours in the presence of calcium hydride (10 g). It was then distilled under reduced pressure, and stored under argon. Racemic and L-lactide (Aldrich) were recrystallized from the crystalline commercially available forms and dried under vacuum for 24 hours. The lactide (10% of racemic lactide+90% of L-lactide) was dissolved in 46 ml of toluene, and the solution was then heated at 60° C. for 90 minutes. Polyethylene glycol monomethyl ether (1.54 g, Aldrich, Mₙ=2000 g/mol, viscosity 54,6000 centistokes) was added to 15 mL of dried toluene in a separate round bottom flask and was stirred until complete dissolution. Diethyl zinc (0.35 ml, 1.1 mol/L in toluene) was added via an argon-purged syringe to the polyethylene glycol solution, and the mixture was stirred magnetically for one hour. Then, the zinc containing solution was transferred via a cannula to the lactide containing solution. The mixture was left to react at 60° C. for one hour. Then, 0.2 mL of hydrochloric acid (Fisher, metal grade, 35.5% in water) were added, and the solvent was subsequently evaporated using a rotary evaporator. The diblock was then redissolved in 50 ml of THF. The solution was added to a magnetically stirred beaker containing 250 ml of ether kept at −18° C. The solid was separated from the liquid by filtration over a fritted glass filter (pore size: 10 to 16 microns) and was washed twice with cold ether (2*50 mL). It was then dried under vacuum (residual pressure<100 microtorrs) for 12 hours at room temperature. 7.8 g of diblock copolymer were collected.

Analysis:

The solid was analyzed by gel permeation chromatography (GPC), nuclear magnetic resonance of the proton (¹H NMR).

GPC

The GPC instrument was constituted of an isocratic HPLC pump Waters 515, a refractometric detector Waters 2414, an autosampler Waters 717 plus, a monowavelength UV-VIS detector Waters 486, an oven (Waters temperature control module). The acquisition and treatment software was Millenium 32. The instrument was equipped with a series of four columns from Polymer Laboratories (PLgel 5 um 100A, PLgel 3 um 100A, PLgel 5 um MIXED-C, PLgel 5 um MIXED-C). Tetrahydrofuran was used as eluent at 35° C. and the flow rate was set at 1 mL/min. The calibration was done using polystyrene standards ranging in molecular weight from 695 to 361,000 g/mol. The molecular weight (polystyrene standard) of the solid is Mₙ=10478 g/mol, M_{w}/Mₙ=1.172. The chromatogram indicates that there is no more residual polyethylene glycol left, indicating that a diblock copolymer has been obtained.

NMR

¹H NMR analysis was realized on a Varian Mercury 400 MHz NMR using CDCl₃ as solvent (relaxation delay of one second, 45 degree pulse, 16 repetitions). TMS was used as reference.

¹H NMR (400 MHz)

| solvent | | CDCl₃ (δ, |
|---|---|---|
| signal | | ppm) |
| PEG | CH₂ | 3.7 |
| PLA | CH | 5.17 |
| | CH₃ | 1.57 |

Experiment 1.3 Diblock Copolymer Poly(ethylene glycol-b-lactide)

| | weight | volume |
|---|---|---|
| Reagent | (g) | (mL) |
| Et₂Zn | | 0.26 |
| PEG | 1.16 | |
| Rac-Lactide | 5.79 | |
| toluene (dried) | | 46.5 |
| HCl | | 0.15 |
| THF | | 50 |
| diethyl ether | | 250 |

This experiment was realized using standard Schlenk techniques required to manipulate air-sensitive reagents. The toluene (1 L), used in this experiment, was magnetically stirred for 24 hours in the presence of calcium hydride (10 g). Racemic lactide (rac-lactide, Aldrich) was recrystallized from the commercially available form and dried under vacuum for 24 hours. The lactide was dissolved in 35 ml of toluene, and the solution was then heated at 60° C. for one hour and half. Polyethylene glycol monomethyl ether (1.16 g, Aldrich, Mₙ=2000 g/mol, viscosity 54,6000 centistokes) was added to 11.5 mL of dried toluene in a separate round bottom flask and was stirred until complete dissolution. Diethyl zinc (0.26 ml, Aldrich, 1.1 mol/L in toluene) was added via an argon-flushed syringe to the polyethylene glycol solution. Then, the zinc containing solution was transferred via a cannula to the lactide containing solution. The mixture was left to react at 60° C. for one hour. Then, 0.15 mL of hydrochloric acid (Fisher, metal grade, 35.5% in water) were added, and the solvent was subsequently evaporated using a rotary evaporator. The diblock was then redissolved in 50 ml of THF. The solution was added to a magnetically stirred beaker containing 250 ml of ether kept at −18° C. The solid was separated from the liquid by filtration over a fritted glass filter (pore size: 10 to 16 microns) and was washed twice with cold ether (2*50 mL). It was then dried under vacuum (residual pressure<100 microtorrs) for 12 hours at room temperature. 2.92 g of diblock copolymer were collected.

Analysis:

The solid was analyzed by gel permeation chromatography (GPC) and nuclear magnetic resonance of the proton (¹H NMR).

GPC

The GPC instrument was constituted of an isocratic HPLC pump Waters 515, a refractometric detector Waters 2414, an autosampler Waters 717 plus, a monowavelength UV-VIS detector Waters 486, an oven (Waters temperature control module). The acquisition and treatment software was Millenium 32. The instrument was equipped with a series of four columns from Polymer Laboratories (PLgel 5 um 100A, PLgel 3 um 100A, PLgel 5 um MIXED-C, PLgel 5 um MIXED-C). Tetrahydrofuran was used as eluent at 35° C. and the flow rate was set at 1 mL/min. The calibration was done using polystyrene standards ranging in molecular weight from 695 to 361,000 g/mol. The molecular weight (polystyrene standard) of the solid is Mₙ=12083 g/mol, M_{w}/Mₙ=1.175. The chromatogram indicates that there is no more residual polyethylene glycol left, indicating that a diblock copolymer has been obtained.

NMR

1H NMR analysis was realized on a Varian Mercury 400 MHz NMR using CDCl₃ as solvent (Relaxation delay of one second, 45 degree pulse, 16 repetitions). TMS was used as reference.

¹H NMR (400 MHz)

| solvent | | CDCl₃ (δ, |
|---|---|---|
| signal | | ppm) |
| PEG | CH₂ | 3.6 |
| PLA | CH | 5.15 |
| | CH₃ | 1.5 |

Experiment 1.4 Diblock Copolymer Poly(ethylene glycol-b-lactide)

| | weight | volume |
|---|---|---|
| Reagent | (g) | (mL) |
| Et₂Zn | | 0.46 |
| PEG | 2.05 | |
| L-Lactide | 10.25 | |
| toluene (dried) | | 82 |
| HCl | | 0.27 |
| THF | | 100 |
| diethyl ether | | 500 |

This experiment was realized using standard Schlenk techniques required to manipulate air-sensitive reagents. The toluene (1 L), used in this experiment, was magnetically stirred for 24 hours in the presence of calcium hydride (10 g). It was then distilled under reduced pressure, and stored under argon. L-lactide (Aldrich) was recrystallized and dried under vacuum for 24 hours. The L-lactide was dissolved in 60 ml of toluene, and the solution was then heated at 60° C. for one hour and half. Polyethylene glycol monomethyl ether (2.05 g, Aldrich, Mₙ=2000 g/mol, viscosity 54,6000 centistokes) was added to 20 mL of dried toluene in a separate round bottom flask and was stirred until complete dissolution. Diethyl zinc (0.45 ml, Aldrich, 1.1 mol/L in toluene) was added via an argon-flushed syringe to the polyethylene glycol solution, and the mixture was stirred magnetically for one hour. Then, the zinc containing solution was transferred via a cannula to the lactide containing solution. The mixture was left to react at 60° C. for one hour. Then, 0.26 mL of hydrochloric acid (Fisher, metal grade, 35.5% in water) was added, and the solvent was subsequently evaporated using a rotary evaporator. The diblock was then redissolved in 50 ml of THF. The solution was added to a magnetically stirred beaker containing 250 ml of ether kept at −18° C. The solid was separated from the liquid by filtration over a fritted glass filter (pore size: 10 to 16 microns) and was washed twice with cold ether (2*50 mL). It was then dried under vacuum (residual pressure<100 microtorrs) for 12 hours at room temperature. 9.83 g of diblock copolymer were collected.

Analysis:

The solid was analyzed by gel permeation chromatography (GPC) and nuclear magnetic resonance of the proton (¹H NMR).

GPC

The GPC instrument was constituted of an isocratic HPLC pump Waters 515, a refractometric detector Waters 2414, an autosampler Waters 717 plus, a monowavelength UV-VIS detector Waters 486, an oven (Waters temperature control module). The acquisition and treatment software was Millenium 32. The instrument was equipped with a series of three columns from Polymer Laboratories (PLgel 5 um 100A, PLgel 5 um MIXED-C, PLgel 5 um MIXED-C). Tetrahydrofuran was used as eluent at 35° C. and the flow rate was set at 1 mL/min. The calibration was done using polystyrene standards ranging in molecular weight from 695 to 361,000 g/mol. The molecular weight (polystyrene standard) of the solid is Mₙ=22807 g/mol, M_{w}/Mₙ=1.15. The chromatogram indicates that there is no more residual polyethylene glycol left, indicating that a diblock copolymer has been obtained.

NMR

1H NMR analysis was realized on a Varian Mercury 400 MHz NMR using CDCl3 as solvent (Relaxation delay of two second, 45 degree pulse, 16 repetitions). TMS was used as reference.

¹H NMR (400 MHz)

| solvent | | CDCl₃ (δ, |
|---|---|---|
| signal | | ppm) |
| PEG | CH₂ | 3.65 |
| PLA | CH | 5.15 |
| | CH₃ | 1.58 |

Experiment 1.5 Diblock Copolymer Poly(ethylene glycol-b-lactide)

| | weight | volume |
|---|---|---|
| Reagent | (g) | (mL) |
| Et₂Zn | | 0.35 |
| PEG | 1.53 | |
| L-Lactide | 11.46 | |
| toluene (dried) | | 84 |
| HCl | | 0.2 |
| THF | | 200 |
| diethyl ether | | 1000 |

This experiment was realized using standard Schlenk techniques required to manipulate air-sensitive reagents. The toluene (1 L), used in this experiment, was magnetically stirred for 24 hours in the presence of calcium hydride (10 g). It was then distilled under reduced pressure, and stored under argon. L-lactide (Aldrich) was recrystallized and dried under vacuum for 24 hours. The L-lactide was dissolved in 60 ml of toluene, and the solution was then heated at 60° C. for one hour and half. Polyethylene glycol monomethyl ether (1.53 g, Aldrich, Mₙ=2000 g/mol, viscosity 54,6000 centistokes) was added to 20 mL of dried toluene in a separate round bottom flask and was stirred until complete dissolution. Diethyl zinc (0.35 ml, Aldrich, 1.1 mol/L in toluene) was added via an argon-flushed syringe to the polyethylene glycol solution, and the mixture was stirred magnetically for one hour. Then, the zinc containing solution was transferred via a cannula to the lactide containing solution. The mixture was left to react at 60° C. for one hour. Then, 0.2 mL of hydrochloric acid (Fisher, metal grade, 35.5% in water) was added, and the solvent was subsequently evaporated using a rotary evaporator. The diblock was then redissolved in 200 ml of THF. The solution was added to a magnetically stirred beaker containing 1 L of ether kept at −18° C. The solid was separated from the liquid by filtration over a fritted glass filter (pore size: 10 to 16 microns) and was washed twice with cold ether (2*200 mL). It was then dried under vacuum (residual pressure<100 microtorrs) for 12 hours at room temperature. 10.17 g of diblock copolymer were collected.

Analysis:

The solid was analyzed by gel permeation chromatography (GPC), nuclear magnetic resonance of the proton (¹H NMR).

GPC

The GPC instrument was constituted of an isocratic HPLC pump Waters 515, a refractometric detector Waters 2414, an autosampler Waters 717 plus, a monowavelength UV-VIS detector Waters 486, an oven (Waters temperature control module). The acquisition and treatment software was Millenium 32. The instrument was equipped with a series of three columns from Polymer Laboratories (PLgel 5 um 100A and 2 PLgel 5 um MIXED-C). Tetrahydrofuran was used as eluent at 35° C. and the flow rate was set at 1 mL/min. The calibration was done using polystyrene standards ranging in molecular weight from 695 to 361,000 g/mol. The molecular weight (polystyrene standard) of the solid is Mₙ=21334 g/mol, M_{w}/Mₙ=1.19. The chromatogram indicates that there is no more residual polyethylene glycol left, indicating that a diblock copolymer has been obtained.

NMR

1H NMR analysis was realized on a Varian Mercury 400 MHz NMR using CDCl3 as solvent (Relaxation delay of one second, 45 degree pulse, 16 repetitions). TMS was used as reference.

¹H NMR (400 MHz)

| solvent | | CDCl₃ (δ, |
|---|---|---|
| signal | | ppm) |
| PEG | CH₂ | 3.65 |
| PLA | CH | 5.17 |
| | CH₃ | 1.59 |

Experiment 1.6 Diblock Copolymer Poly(ethylene glycol-b-lactide)

| | weight | volume |
|---|---|---|
| Reagent | (g) | (mL) |
| Et₂Zn | | 0.23 |
| PEG | 1.01 | |
| L-Lactide | 7.55 | |
| toluene (dried) | | 55 |
| Methanol | | 20 |
| THF | | 170 |
| diethyl ether | | 850 |

This experiment was realized using standard Schlenk techniques required to manipulate air-sensitive reagents. The toluene (1 L), used in this experiment, was magnetically stirred for 24 hours in the presence of calcium hydride (10 g). It was then distilled under reduced pressure, and stored under argon. L-lactide (Aldrich) was recrystallized and dried under vacuum for 24 hours. The L-lactide was dissolved in 60 ml of toluene, and the solution was then heated at 60° C. for one hour and half. Polyethylene glycol monomethyl ether (1.53 g, Aldrich, Mₙ=2000 g/mol, viscosity 54,6000 centistokes) was added to 20 mL of dried toluene in a separate round bottom flask and was stirred until complete dissolution. Diethyl zinc (0.23 ml, Aldrich, 1.1 mol/L in toluene) was added via an argon-flushed syringe to the polyethylene glycol solution, and the mixture was stirred magnetically for one hour. Then, the zinc containing solution was transferred via a cannula to the lactide containing solution. The mixture was left to react at 60° C. for one hour. Then, 0.2 mL of hydrochloric acid (Fisher, metal grade, 35.5% in water) was added, and the solvent was subsequently evaporated using a rotary evaporator. The diblock was then redissolved in 170 ml of THF. The solution was added to a magnetically stirred beaker containing 850 ml of ether kept at −18° C. The solid was separated from the liquid by filtration over a fritted glass filter (pore size: 10 to 16 microns) and was washed twice with cold ether (2*100 mL). It was then dried under vacuum (residual pressure<100 microtorrs) for 12 hours at room temperature. 5.09 g of diblock copolymer were collected.

Analysis:

The solid was analyzed by gel permeation chromatography (GPC) and nuclear magnetic resonance of the proton (¹H NMR).

GPC

The GPC instrument was constituted of an isocratic HPLC pump Waters 515, a refractometric detector Waters 2414, an autosampler Waters 717 plus, a monowavelength UV-VIS detector Waters 486, an oven (Waters temperature control module). The acquisition and treatment software was Millenium 32. The instrument was equipped with a series of three columns from Polymer Laboratories (PLgel 5 um 100A, PLgel 5 um MIXED-C, PLgel 5 um MIXED-C). Tetrahydrofuran was used as eluent at 35° C. and the flow rate was set at 1 mL/min. The calibration was done using polystyrene standards ranging in molecular weight from 695 to 361,000 g/mol. The molecular weight (polystyrene standard) of the solid is Mₙ=14783 g/mol, M_{w}/Mₙ=1.35. The chromatogram indicates that there is no more residual polyethylene glycol left, indicating that a diblock copolymer has been obtained.

NMR

1H NMR analysis was realized on a Varian Mercury 400 MHz NMR using CDCl3 as solvent (Relaxation delay of one second, 45 degree pulse, 16 repetitions). TMS was used as reference.

¹H NMR (400 MHz)

| solvent | | CDCl₃ (δ, |
|---|---|---|
| signal | | ppm) |
| PEG | CH₂ | 3.65 |
| PLA | CH | 5.17 |
| | CH₃ | 1.58 |

Experiment 1.7 Diblock Copolymer Poly(ethylene glycol-b-lactide)

| | weight | volume |
|---|---|---|
| Reagent | (g) | (mL) |
| Et₂Zn | | 0.33 |
| PEG | 1.44 | |
| L-Lactide | 10.78 | |
| toluene (dried) | | 68.4 |
| Methanol | | 392 |
| THF | | 100 |
| diethyl ether | | 500 |

This experiment was realized using standard Schlenk techniques required to manipulate air-sensitive reagents. The toluene (1 L), used in this experiment, was magnetically stirred for 24 hours in the presence of calcium hydride (10 g). It was then distilled under reduced pressure, and stored under argon. L-lactide (Aldrich) was recrystallized and dried under vacuum for 24 hours. The L-lactide was dissolved in 60 ml of toluene, and the solution was then heated at 60° C. for one hour and half. Polyethylene glycol monomethyl ether (1.44 g, Aldrich, Mₙ=2000 g/mol, viscosity 54,6000 centistokes) was added to 14.4 mL of dried toluene in a separate round bottom flask and was stirred until complete dissolution. Diethyl zinc (0.33 ml, Aldrich, 1.1 mol/L in toluene) was added via an argon-flushed syringe to the polyethylene glycol solution, and the mixture was stirred magnetically for one hour. Then, the zinc containing solution was transferred via a cannula to the lactide containing solution. The mixture was left to react at 60° C. for one hour. Then, the solution was poured in 392 mL of Methanol (EMD, HPLC grade), and the solvent was subsequently evaporated using a rotary evaporator. The diblock was then redissolved in 100 ml of THF. The solution was added to a magnetically stirred beaker containing 500 ml of ether kept at −18° C. The solid was separated from the liquid by filtration over a fritted glass filter (pore size: 10 to 16 microns) and was washed twice with cold ether (2*50 mL). It was then dried under vacuum (residual pressure<100 microtorrs) for 12 hours at room temperature. 4.87 g of diblock copolymer were collected.

Analysis:

The solid was analyzed by gel permeation chromatography (GPC) and nuclear magnetic resonance of the proton (¹H NMR).

GPC

The GPC instrument was constituted of an isocratic HPLC pump Waters 515, a refractometric detector Waters 2414, an autosampler Waters 717 plus, a monowavelength UV-VIS detector Waters 486, an oven (Waters temperature control module). The acquisition and treatment software was Millenium 32. The instrument was equipped with a series of three columns from Polymer Laboratories (PLgel 5 um 100A, PLgel 5 um MIXED-C, PLgel 5 um MIXED-C). Tetrahydrofuran was used as eluent at 35° C. and the flow rate was set at 1 mL/min. The calibration was done using polystyrene standards ranging in molecular weight from 695 to 361,000 g/mol. The molecular weight (polystyrene standard) of the solid is Mₙ=5247 g/mol, M_{w}/Mₙ=1.39. The chromatogram indicates that there is no more residual polyethylene glycol left, indicating that a diblock copolymer has been obtained.

NMR

1H NMR analysis was realized on a Varian Mercury 400 MHz NMR using CDCl3 as solvent (Relaxation delay of one second, 45 degree pulse, 16 repetitions). TMS was used as reference.

¹H NMR (400 MHz)

| solvent | | CDCl₃ (δ, |
|---|---|---|
| signal | | ppm) |
| PEG | CH₂ | 3.61 |
| PLA | CH | 5.12 |
| | CH₃ | 1.45 |

Experiment 1.8 Diblock Copolymer Poly(ethylene glycol-b-lactide)

| | weight | Volume |
|---|---|---|
| Reagent | (g) | (mL) |
| Et₂Zn | | 0.45 |
| PEG | 0.75 | |
| L-Lactide | 10.08 | |
| toluene (dried) | | 69 |
| Methanol | | 350 |
| THF | | 150 |
| diethyl ether | | 750 |

This experiment was realized using standard Schlenk techniques required to manipulate air-sensitive reagents. The toluene (1 L), used in this experiment, was magnetically stirred for 24 hours in the presence of calcium hydride (10 g). It was then distilled under reduced pressure, and stored under argon. L-lactide (Aldrich) was recrystallized and dried under vacuum for 24 hours. The L-lactide was dissolved in 64 ml of toluene, and the solution was then heated at 60° C. for one hour. Polyethylene glycol monomethyl ether (0.75 g, Aldrich, Mₙ=750 g/mol, viscosity 10,500 centistokes) was added to 9 mL of dried toluene in a separate round bottom flask and was stirred until complete dissolution. Diethyl zinc (0.45 ml, Aldrich, 1.1 mol/L in toluene) was added via an argon-flushed syringe to the polyethylene glycol solution, and the mixture was stirred magnetically for one hour. Then, the zinc containing solution was transferred via a cannula to the lactide containing solution. The mixture was left to react at 60° C. for 40 minutes. Then, the solution was poured in 350 mL of Methanol (EMD, HPLC grade), and the solvent was subsequently evaporated using a rotary evaporator. The diblock was then redissolved in 100 ml of THF. The solution was added to a magnetically stirred beaker containing 500 ml of ether kept at −18° C. The solid was separated from the liquid by filtration over a fritted glass filter (pore size: 10 to 16 microns) and was washed twice with cold ether (2*50 mL). It was then dried under vacuum (residual pressure<100 microtorrs) for 12 hours at room temperature. 9.61 g of diblock copolymer were collected.

Analysis:

The solid was analyzed by gel permeation chromatography (GPC) and nuclear magnetic resonance of the proton (¹H NMR).

GPC

The GPC instrument was constituted of an isocratic HPLC pump Waters 515, a refractometric detector Waters 414, an autosampler Waters 717 plus, a monowavelength UV-VIS detector Waters 486, an oven (Waters temperature control module). The acquisition and treatment software was Millenium 32. The instrument was equipped with a series of three columns from Waters (2 Styragel HR4E and Eurogel HE145). Tetrahydrofuran was used as eluent at 45° C. and the flow rate was set at 0.7 mL/min. The calibration was done using polystyrene standards ranging in molecular weight from 695 to 361,000 g/mol. The molecular weight (polystyrene standard) of the solid is Mₙ=22393 g/mol, M_{w}/Mₙ=1.1. The chromatogram indicates that there is no more residual polyethylene glycol left, indicating that a diblock copolymer has been obtained.

NMR

¹H NMR analysis was realized on a Varian Mercury 400 MHz NMR using CDCl3 as solvent (Relaxation delay of one second, 45 degree pulse, 16 repetitions). TMS was used as reference.

¹H NMR (400 MHz)

| Solvent signal | | CDCl₃ (δ, ppm) |
|---|---|---|
| PEG | CH₂ | 3.64 |
| PLA | CH | 5.16 |
| | CH₃ | 1.58 |

Experiment 1.9 Diblock Copolymer Poly(ethylene glycol-b-lactide)

| | weight | Volume |
|---|---|---|
| Reagent | (g) | (mL) |
| Et₂Zn | | 1.36 |
| PEG | 2.25 | |
| L-Lactide | 15.12 | |
| toluene (dried) | | 117 |
| Methanol | | 585 |
| THF | | 150 |
| diethyl ether | | 750 |

This experiment was realized using standard Schlenk techniques required to manipulate air-sensitive reagents. The toluene (1 L), used in this experiment, was magnetically stirred for 24 hours in the presence of calcium hydride (10 g). It was then distilled under reduced pressure, and stored under argon. L-lactide (Aldrich) was recrystallized and dried under vacuum for 24 hours. The L-lactide was dissolved in 90 ml of toluene, and the solution was then heated at 60° C. for one hour. Polyethylene glycol monomethyl ether (2.25 g, Aldrich, Mₙ=750 g/mol, viscosity 10,500 centistokes) was added to 27 mL of dried toluene in a separate round bottom flask and was stirred until complete dissolution. Diethyl zinc (0.45 ml, Aldrich, 1.1 mol/L in toluene) was added via an argon-flushed syringe to the polyethylene glycol solution, and the mixture was stirred magnetically for one hour. Then, the zinc containing solution was transferred via a cannula to the lactide containing solution. The mixture was left to react at 60° C. for 40 minutes. Then, the solution was poured in 585 mL of Methanol (EMD, HPLC grade), and the solvent was subsequently evaporated using a rotary evaporator. The diblock was then redissolved in 150 ml of THF. The solution was added to a magnetically stirred beaker containing 500 ml of ether kept at −18° C. The solid was separated from the liquid by filtration over a fritted glass filter (pore size: 10 to 16 microns) and was washed twice with cold ether (2*100 mL). It was then dried under vacuum (residual pressure<100 microtorrs) for 12 hours at room temperature. 14.72 g of diblock copolymer were collected.

Analysis:

The solid was analyzed by gel permeation chromatography (GPC), nuclear magnetic resonance of the proton (¹H NMR) and differential scanning calorimetry (DSC).

GPC

The GPC instrument was constituted of an isocratic HPLC pump Waters 515, a refractometric detector Waters 414, an autosampler Waters 717 plus, a monowavelength UV-VIS detector Waters 486, an oven (Waters temperature control module). The acquisition and treatment software was Millenium 32. The instrument was equipped with a series of three columns from Waters (2 Styragel HR4E and Eurogel HE145). Tetrahydrofuran was used as eluent at 45° C. and the flow rate was set at 0.7 mL/min. The calibration was done using polystyrene standards ranging in molecular weight from 695 to 361,000 g/mol. The molecular weight (polystyrene standard) of the solid is Mₙ=8959 g/mol, M_{w}/Mₙ=1.29. The chromatogram indicates that there is no more residual polyethylene glycol left, indicating that a diblock copolymer has been obtained.

NMR

1H NMR analysis was realized on a Varian Mercury 400 MHz NMR using CDCl3 as solvent (Relaxation delay of one second, 45 degree pulse, 16 repetitions). TMS was used as reference.

¹H NMR (400 MHz)

| solvent | | CDCl₃ (δ, |
|---|---|---|
| signal | | ppm) |
| PEG | CH₂ | 3.64 |
| PLA | CH | 5.16 |
| | CH₃ | 1.58 |

Experiment 1.10 Diblock Copolymer Poly(ethylene glycol-b-lactide)

| | weight | volume |
|---|---|---|
| Reagent | (g) | (mL) |
| Et₂Zn | | 0.45 |
| PEG | 2.00 | |
| L-Lactide | 5.04 | |
| toluene (dried) | | 50 |
| Methanol | | 250 |

This experiment was realized using standard Schlenk techniques required to manipulate air-sensitive reagents. The toluene (1 L), used in this experiment, was magnetically stirred for 24 hours in the presence of calcium hydride (10 g). It was then distilled under reduced pressure, and stored under argon. L-lactide (Aldrich) was recrystallized and dried under vacuum for 24 hours. The L-lactide was dissolved in 30 ml of toluene, and the solution was then heated at 60° C. for one hour. Polyethylene glycol monomethyl ether (2.00 g, Aldrich, Mn=2000 g/mol, viscosity 54,600 centistokes) was added to 20 mL of dried toluene in a separate round bottom flask and was stirred until complete dissolution. Diethyl zinc (0.45 ml, Aldrich, 1.1 mol/L in toluene) was added via an argon-flushed syringe to the polyethylene glycol solution, and the mixture was stirred magnetically for one hour. Then, the zinc containing solution was transferred via a cannula to the lactide containing solution. The mixture was left to react at 60° C. for two hours. Then, the solution was poured in 250 mL of methanol (EMD, HPLC grade), and the solvent was subsequently evaporated using a rotary evaporator. The diblock was then redissolved in 150 ml of THF. The solution was added to a magnetically stirred beaker containing 500 ml of ether kept at −18° C. The solid was separated from the liquid by filtration over a fritted glass filter (pore size: 10 to 16 microns) and was washed twice with cold ether (2*50 mL). It was then dried under vacuum (residual pressure<100 microtorrs) for 12 hours at room temperature. 3.67 g of diblock copolymer were collected.

Analysis:

The solid was analyzed by gel permeation chromatography (GPC) and nuclear magnetic resonance of the proton (¹H NMR).

GPC

The GPC instrument was constituted of an isocratic HPLC pump Waters 515, a refractometric detector Waters 414, an autosampler Waters 717 plus, a monowavelength UV-VIS detector Waters 486, an oven (Waters temperature control module). The acquisition and treatment software was Millenium 32. The instrument was equipped with a series of three columns from Waters (2 Styragel HR4E and Eurogel HE145). Tetrahydrofuran was used as eluent at 45° C. and the flow rate was set at 0.7 mL/min. The calibration was done using polystyrene standards ranging in molecular weight from 695 to 361,000 g/mol. The molecular weight (polystyrene standard) of the solid is Mₙ=3385 g/mol, M_{w}/Mₙ=1.09. The chromatogram indicates that there is no more residual polyethylene glycol left, indicating that a diblock copolymer has been obtained.

NMR

1H NMR analysis was realized on a Varian Mercury 400 MHz NMR using CDCl3 as solvent (Relaxation delay of one second, 45 degree pulse, 16 repetitions). TMS was used as reference.

¹H NMR (400 MHz)

| solvent | | CDCl₃ (δ, |
|---|---|---|
| signal | | ppm) |
| PEG | CH₂ | 3.64 |
| PLA | CH | 5.12 |
| | CH₃ | 1.55 |

Experiment 1.11 Diblock Copolymer Poly(ethylene glycol-b-lactide)

| | weight | volume |
|---|---|---|
| Reagent | (g) | (mL) |
| Et₂Zn | | 1.21 |
| PEG | 2.01 | |
| L-Lactide | 8.06 | |
| toluene (dried) | | 1000 |
| Methanol | | 500 |

This experiment was realized using standard Schlenk techniques required to manipulate air-sensitive reagents. The toluene (1 L), used in this experiment, was magnetically stirred for 24 hours in the presence of calcium hydride (10 g). It was then distilled under reduced pressure, and stored under argon. L-lactide (Aldrich) was recrystallized and dried under vacuum for 24 hours. The L-lactide was dissolved in 50 ml of toluene, and the solution was then heated at 60° C. for one hour. Polyethylene glycol monomethyl ether (2.01 g, Aldrich, Mₙ=750 g/mol, viscosity 10,500 centistokes) was added to 50 mL of dried toluene in a separate round bottom flask and was stirred until complete dissolution. Diethyl zinc (1.21 ml, Aldrich, 1.1 mol/L in toluene) was added via an argon-flushed syringe to the polyethylene glycol solution, and the mixture was stirred magnetically for one hour. Then, the zinc containing solution was transferred via a cannula to the lactide containing solution. The mixture was left to react at 80° C. for two hours. Then, the solution was poured in 500 mL of methanol (EMD, HPLC grade), and the solvent was subsequently evaporated using a rotary evaporator. The diblock was then redissolved in 150 ml of THF. The solution was added to a magnetically stirred beaker containing 500 ml of ether kept at −18° C. The solid was separated from the liquid by filtration over a fritted glass filter (pore size: 10 to 16 microns) and was washed twice with cold ether (2*50 mL). It was then dried under vacuum (residual pressure<100 microtorrs) for 12 hours at room temperature. 3.68 g of diblock copolymer were collected.

Analysis:

The solid was analyzed by gel permeation chromatography (GPC) and nuclear magnetic resonance of the proton (¹H NMR).

GPC

The GPC instrument was constituted of an isocratic HPLC pump Waters 515, a refractometric detector Waters 414, an autosampler Waters 717 plus, a monowavelength UV-VIS detector Waters 486, an oven (Waters temperature control module). The acquisition and treatment software was Millenium 32. The instrument was equipped with a series of three columns from Waters (2 Styragel HR4E and Eurogel HE145). Tetrahydrofuran was used as eluent at 45° C. and the flow rate was set at 0.7 mL/min. The calibration was done using polystyrene standards ranging in molecular weight from 695 to 361,000 g/mol. The molecular weight (polystyrene standard) of the solid is Mₙ=2423 g/mol, M_{w}/Mₙ=1.26. The chromatogram indicates that there is no more residual polyethylene glycol left, indicating that a diblock copolymer has been obtained.

NMR

1H NMR analysis was realized on a Varian Mercury 400 MHz NMR using CDCl3 as solvent (Relaxation delay of one second, 45 degree pulse, 16 repetitions). TMS was used as reference.

¹H NMR (400 MHz)

| solvent | | CDCl₃ (δ, |
|---|---|---|
| signal | | ppm) |
| PEG | CH₂ | 3.65 |
| PLA | CH | 5.16 |
| | CH₃ | 1.59 |

Experiment 1.12 Diblock Copolymer Poly(ethylene glycol-b-lactide)

| | weight | Volume |
|---|---|---|
| Reagent | (g) | (mL) |
| Et₂Zn | | 0.5 |
| PEG | 0.30 | |
| L-Lactide | 5.59 | |
| toluene (dried) | | 40 |
| Methanol | | 200 |

This experiment was realized using standard Schlenk techniques required to manipulate air-sensitive reagents. The toluene (1 L), used in this experiment, was magnetically stirred for 24 hours in the presence of calcium hydride (10 g). It was then distilled under reduced pressure, and stored under argon. L-lactide (Aldrich) was recrystallized and dried under vacuum for 24 hours. The L-lactide was dissolved in 30 ml of toluene, and the solution was then heated at 80° C. for one hour. Polyethylene glycol monomethyl ether (0.30 g, Aldrich, Mₙ=350 g/mol, viscosity 4,100 centistokes) was added to 10 mL of dried toluene in a separate round bottom flask and was stirred until complete dissolution. Diethyl zinc (0.50 ml, Aldrich, 1.1 mol/L in toluene) was added via an argon-flushed syringe to the polyethylene glycol solution, and the mixture was stirred magnetically for one hour. Then, the zinc containing solution was transferred via a cannula to the lactide containing solution. The mixture was left to react at 80° C. for four hours. Then, the solution was poured in 200 mL of methanol (EMD, HPLC grade), and the solvent was subsequently evaporated using a rotary evaporator. The solid was separated from the liquid by filtration over a fritted glass filter (pore size: 10 to 16 microns) and was washed twice with cold ether (2*50 mL). It was then dried under vacuum (residual pressure<100 microtorrs) for 12 hours at room temperature. 5.14 g of diblock copolymer were collected.

Analysis:

The solid was analyzed by gel permeation chromatography (GPC) and nuclear magnetic resonance of the proton (¹H NMR).

GPC

The GPC instrument was constituted of an isocratic HPLC pump Waters 515, a refractometric detector Waters 414, an autosampler Waters 717 plus, a monowavelength UV-VIS detector Waters 486, an oven (Waters temperature control module). The acquisition and treatment software was Millenium 32. The instrument was equipped with a series of three columns from Waters (2 Styragel HR4E and Eurogel HE145). Tetrahydrofuran was used as eluent at 45° C. and the flow rate was set at 0.7 mL/min. The calibration was done using polystyrene standards ranging in molecular weight from 695 to 361,000 g/mol. The molecular weight (polystyrene standard) of the solid is Mₙ=9175 g/mol, M_{w}/Mₙ=1.40. The chromatogram indicates that there is no more residual polyethylene glycol left, indicating that a diblock copolymer has been obtained.

NMR

1H NMR analysis was realized on a Varian Mercury 400 MHz NMR using CDCl3 as solvent (Relaxation delay of one second, 45 degree pulse, 16 repetitions). TMS was used as reference.

¹H NMR (400 MHz)

| solvent | | CDCl₃ (δ, |
|---|---|---|
| signal | | ppm) |
| PEG | CH₂ | 3.64 |
| PLA | CH | 5.17 |
| | CH₃ | 1.57 |

Experiment 2.1 Triblock Copolymer Poly(glutamic acid-b-lactide-b-ethylene glycol)

| Rgt | m (g) | V (mL) |
|---|---|---|
| PEG-PLA | 0.5034 | |
| PGlu | 1.1047 | |
| DCC | 0.0368 | |
| N- | 0.0199 | |
| hydroxysuccinimide | | |
| N-methyl | | 8 |
| pyrrolidinone | | |

Polyglutamic acid (1.1047 g, MW=12900 g/mol, prepared according to the procedure described in by H. Kricheldorf, α-aminoacid-N-carboxy anhydride and related heterocycles, Springer-Verlag, 1987 page 88), N-hydroxysuccinimide (8 mg), and the polymer described in experiment 1.1 were suspended in N-methylpyrrolidinone (NMP, 7.8 ml). 36.8 mg of dicyclohexyl carbodiimide (DCC) was dissolved in NMP, in another round bottom flask. Then the two liquids were mixed together, and stirred at 55° C. for 2 hours. Then 40 ml of ultrapure water was added to the mixture, and the suspension (48 ml) was then ultrafiltered in an Amicon Stirred Ultrafiltration cell of 10 ml, and using a polyethersulfone membrane with a cutoff of 500,000 g/mol. The pressure used during the ultrafiltration was 30 psi, and 100 ml of water were used for the experiment. At the end of the ultrafiltration experiment, the polymer suspension was concentrated down to a volume of 5 ml. The concentrated solution was freeze dried to afford an off-white solid.

Analysis:

¹H NMR (400 MHz)

| | | Trifluoroacetic |
|---|---|---|
| solvent signal | | acid (δ, ppm) |
| PEG | CH₂ | 4.03 |
| PLA | CH | 5.51 |
| | CH₃ | 1.78 |
| Pglu | CH | 4.95 |
| | (CH—)CH₂ₐ | 2.28 |
| | (CH—)CH_{2b} | 2.43 |
| | (CO2H—)CH₂ | 2.77 |

Transmission Electron Micrograph (TEM)

Suspensions were prepared by suspending the polymer prepared in Experiment number 2.1 in Hepes buffer (25 mM, pH=8.2) at a concentration in weight of 0.01% in weight. After 20 minutes of sonication, a drop of the suspension was deposited on a gold grid (Formvar carbon support film on specimen grid, Mesh 400). Then another drop of staining agent (uracyl acetate, 0.01% in weight) was added. The electron microscope used was a JEOL 100S TEM with an accelerating voltage of 80 KV. A 2 s exposure time was used to take cliches of the polymer, showing the presence of vesicles (number average diameter=250 nm).

Dynamic Light Scattering (QELS)

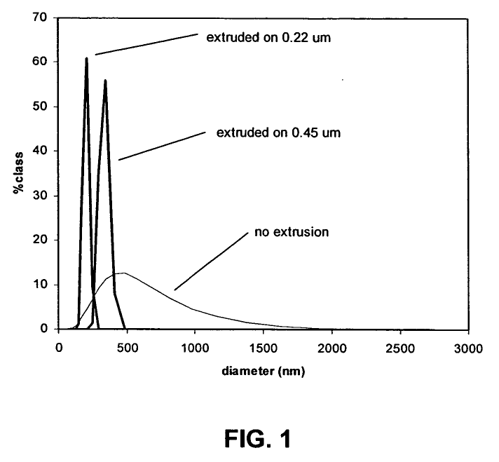

Vesicle size was also assessed by dynamic light scattering, using a Nanotrac by Microtrac. Suspensions were prepared by suspending the polymer prepared in Experiment number 2.1 in phosphate buffer (300 mOsm, pH=7.2) at a concentration in weight of 1% in weight. The suspension was then sonicated for 10 minutes. The average vesicle diameter, measured by dynamic light scattering was 475 nm with a standard deviation of 232 nm (see FIG. 1). The suspension was then extruded through a 0.45 μm disk filter (Syrfil-MF, membra-FIL mixed cellulose ester from Whatman). The average vesicle diameter, measured by dynamic light scattering was 301 nm with a standard deviation of 33 nm (see FIG. 1). The suspension was then extruded through a 0.22 μm disk filter (Syrfil-MF, membra-FIL mixed cellulose ester from Whatman). The average vesicle diameter, measured by dynamic light scattering was 181 nm with a standard deviation of 18 nm (see FIG. 1). This shows that the size of the vesicles can be tailored by extrusion.

Experiment 2.2 Triblock Copolymer Poly(glutamic acid-b-lactide-b-ethylene glycol)

| Rgt | m (g) | V (mL) |
|---|---|---|
| PEG-PLA | 0.0320 | |
| PGlu | 0.0445 | |
| DCC | 0.0052 | |
| N- | 0.0028 | |
| hydroxysuccinimide | | |
| N-methyl | | 0.84 |
| pyrrolidinone | | |

Polyglutamic acid (0.0445 g, Biochemika, MW comprised between 2000 g/mol and 15000 g/mol), N-hydroxysuccinimide (2.8 mg), and the polymer described in experiment 1.2 were suspended in N-methylpyrrolidinone (NMP, 0.74 ml). 5.2 mg of dicyclohexyl carbodiimide (DCC) was dissolved in NMP, in another round bottom flask. Then the two liquids were mixed together, and stirred at 55° C. for 2 hours. Then 4.2 ml of ultrapure water was added to the mixture, and the suspension (5.04 ml) was freeze dried and the dried using a rotovapor to afford an off-white solid.

Analysis:

¹H NMR (400 MHz)

| | | Trifluoroacetic |
|---|---|---|
| solvent signal | | acid (δ, ppm) |
| PEG | CH₂ | 3.49 |
| PLA | CH | 5.67 |
| | CH₃ | 1.98 |
| Pglu | CH | 5.15 |
| | (CH—)CH₂ₐ | 2.49 |
| | (CH—)CH_{2b} | 2.66 |
| | (CO2H—)CH₂ | 2.97 |

Transmission Electron Micrograph (TEM)

Suspensions were prepared by suspending the polymer prepared in Experiment number 2.2 in phosphate buffer 300 mOsm (pH=7.4) at a concentration in weight of 0.01% in weight. After 20 minutes of sonication, a drop of the suspension was deposited on a gold grid (Formvar carbon support film on specimen grid, Mesh 400). Then another drop of staining agent (uracyl acetate, 0.01% in weight) was added. The electron microscope used was a JEOL 100S TEM with an accelerating voltage of 80 KV. A 2 s exposure time was used to take cliches of the polymer, showing the presence of vesicles (number average diameter=300 nm).

Experiment 2.3 Triblock Copolymer Poly(glutamic acid-b-lactide-b-ethylene glycol)

| Rgt | m (g) | V (mL) |
|---|---|---|
| PEG-PLA | 0.5296 | |
| PGlu | 0.3515 | |
| DCC | 0.0381 | |
| N- | 0.0210 | |
| hydroxysuccinimide | | |
| N-methyl | | 4.76 |
| pyrrolidinone | | |

Polyglutamic acid (0.3515 g, MW=3870 g/mol, prepared according to the procedure described in by H. Kricheldorf, α-aminoacid-N-carboxy anhydride and related heterocycles, Springer-Verlag, 1987 page 88), N-hydroxysuccinimide (21 mg), and the polymer described in experiment 1.3 were suspended in N-methylpyrrolidinone (NMP, 4.26 ml). 38.1 mg of dicyclohexyl carbodiimide (DCC) was dissolved in NMP, in another round bottom flask. Then the two liquids were mixed together, and stirred at 55° C. for 2 hours. The solution was poured in 25 ml of ultrapure water and the suspension (5.04 ml) was centrifuged. The solid was collected by filtration on a fritted glass. The solid was washed by trituration with 25 ml of water, then with 25 mL of methanol and finally with 25 mL of ether. The white solid obtained was dried under vacuum.

Analysis:

¹H NMR (400 MHz)

| | | Trifluoroacetic | D₂O (δ, |
|---|---|---|---|
| | | acid (δ, ppm) | ppm) |
| PEG | CH₂ | 3.97 | 3.54 |
| PLA | CH | 5.45 | 3.94 |
| | CH₃ | 1.72 | 1.16 |
| Pglu | CH | 4.90 | 4.15 |
| | (CH—)CH₂ₐ | 2.03 | 1.76 |
| | (CH—)CH_{2b} | 2.37 | 1.87 |
| | (CO2H—)CH₂ | 2.71 | 2.09 |

Experiment 2.4 Triblock Copolymer Poly(glutamic acid-b-lactide-b-ethylene glycol)

| Rgt | m (g) | V (mL) |
|---|---|---|
| PEG-PLA | 1.1292 | |
| PGlu | 0.4908 | |
| DCC | 0.0523 | |
| N- | 0.0549 | |
| hydroxysuccinimide | | |
| N-methyl | | 16.2 |
| pyrrolidinone | | |

Polyglutamic acid (0.4908 g, MW=3870 g/mol, prepared according to the procedure described in by H. Kricheldorf, α-aminoacid-N-carboxy anhydride and related heterocycles, Springer-Verlag, 1987 page 88), N-hydroxysuccinimide (54.9 mg), and the polymer described in experiment 1.5 were suspended in N-methylpyrrolidinone (NMP, 15.7 ml). 52.3 mg of dicyclohexyl carbodiimide (DCC) was dissolved in NMP, in another round bottom flask. Then the two liquids were mixed together, and stirred at 55° C. for 2 hours. The solution was poured in 79 ml of ultrapure water and the suspension (5.04 ml) was centrifuged. The solid was collected by filtration on a fritted glass. The solid was washed by trituration with 40 ml of water, then with 40 mL of methanol and finally with 40 mL of ether. The white solid obtained was dried under vacuum.

Analysis:

¹H NMR (400 MHz)

| | | | D2O |
|---|---|---|---|
| | | Trifluoroacetic | (δ, |
| solvent signal | | acid (δ, ppm) | ppm) |
| PEG | CH₂ | 3.96 | 3.54 |
| PLA | CH | 5.42 | 3.94 |
| | CH₃ | 1.73 | 1.15 |
| Pglu | CH | 4.88 | 4.14 |
| | (CH—)CH₂ₐ | 2.22 | 1.76 |
| | (CH—)CH_{2b} | 2.37 | 1.87 |
| | (CO2H—)CH₂ | 2.72 | 2.10 |

Experiment 2.5 Triblock Copolymer Poly(glutamic acid-b-lactide-b-ethylene glycol)

| Rgt | m (g) | V (mL) |
|---|---|---|
| PEG-PLA | 0.2499 | |
| PGlu | 0.1137 | |
| DCC | 0.0066 | |
| N- | 0.0069 | |
| hydroxysuccinimide | | |
| N-methyl | | 2.26 |
| pyrrolidinone | | |

Polyglutamic acid (0.1137 g, MW=3870 g/mol, prepared according to the procedure described in by H. Kricheldorf, α-aminoacid-N-carboxy anhydride and related heterocycles, Springer-Verlag, 1987 page 88), N-hydroxysuccinimide (6.9 mg), and the polymer described in experiment 1.7 were suspended in N-methylpyrrolidinone (NMP, 1.76 ml). 6.6 mg of dicyclohexyl carbodiimide (DCC) was dissolved in NMP, in another round bottom flask. Then the two liquids were mixed together, and stirred at 50° C. for 2 hours. The solution was poured in 11.3 ml of ultrapure water and the suspension (13.56 ml) was centrifuged. The solid was collected by filtration on a fritted glass. The solid was washed by trituration with 20 ml of water, then with 20 mL of methanol and finally with 20 mL of ether. The white solid obtained was dried under vacuum.

Analysis:

¹H NMR (400 MHz)

| | | Trifluoroacetic |
|---|---|---|
| solvent signal | | acid (δ, ppm) |
| PEG | CH₂ | 3.99 |
| PLA | CH | 5.45 |
| | CH₃ | 1.75 |
| Pglu | CH | 4.92 |
| | (CH—)CH₂ₐ | 2.25 |
| | (CH—)CH_{2b} | 2.40 |
| | (CO2H—)CH₂ | 2.74 |

Experiment 2.6 Triblock Copolymer Poly(glutamic acid-b-lactide-b-ethylene glycol)

| Rgt | m (g) | V (mL) |
|---|---|---|
| PEG-PLA | 0.1479 | |
| PGlu | 0.1377 | |
| DCC | 0.0074 | |
| N- | 0.0077 | |
| hydroxysuccinimide | | |
| N-methyl | | 1.7 |
| pyrrolidinone | | |

Polyglutamic acid (0.1377 g, MW=3870 g/mol, prepared according to the procedure described in by H. Kricheldorf, α-aminoacid-N-carboxy anhydride and related heterocycles, Springer-Verlag, 1987 page 88), N-hydroxysuccinimide (7.7 mg), and the polymer described in experiment 1.9 were suspended in N-methylpyrrolidinone (NMP, 1.2 ml). 7.4 mg of dicyclohexyl carbodiimide (DCC) was dissolved in NMP, in another round bottom flask. Then the two liquids were mixed together, and stirred at 50° C. for 2 hours. The solution was poured in 8.5 ml of ultrapure water. The solid was collected by filtration on a fritted glass. The solid was washed by trituration with 20 ml of water, then with 20 mL of methanol and finally with 20 mL of ether. The white solid obtained was dried under vacuum.

Analysis:

¹H NMR (400 MHz)

| | | | D2O |
|---|---|---|---|
| | | Trifluoroacetic | (δ, |
| solvent signal | | acid (δ, ppm) | ppm) |
| PEG | CH₂ | 4.18 | 3.67 |
| PLA | CH | 5.64 | 4.06 |
| | CH₃ | 1.94 | 1.28 |
| Pglu | CH | 6.22 | 4.29 |
| | (CH—)CH₂ₐ | 2.64 | 1.89 |
| | (CH—)CH_{2b} | 3.06 | 2.01 |
| | (CO2H—)CH₂ | 3.13 | 2.24 |

Experiment 2.7 Triblock Copolymer Poly(glutamic acid-b-lactide-b-ethylene glycol)

| Rgt | m (g) | V (mL) |
|---|---|---|
| PEG-PLA | 0.0987 | |
| PGlu | 0.0871 | |
| DCC | 0.0027 | |
| N- | 0.0029 | |
| hydroxysuccinimide | | |
| sodium | | |
| N-methyl | | 0.69 |
| pyrrolidinone | | |

Polyglutamic acid (0.0871 g, MW=6450 g/mol, prepared according to the literature) and the polymer described in experiment 1.8 were suspended in N-methyl pyrrolidinone (NMP, 0.6 ml). 2.9 mg of N-hydroxysuccinimide sodium and 7.4 mg of dicyclohexyl carbodiimide (DCC) were dissolved in NMP, in another round bottom flask. Then the two liquids were mixed together, and stirred at 50° C. for 2 hours. The solution was poured in 7.5 ml of ultrapure water. The solid was collected by filtration on a fritted glass. The solid was washed by trituration with 20 ml of water, then with 20 mL of methanol and finally with 20 mL of ether. The white solid obtained was dried under vacuum.

Analysis:

¹H NMR (400 MHz)

| | | Trifluoroacetic | D2O (δ, |
|---|---|---|---|
| solvent signal | | acid (δ, ppm) | ppm) |
| PEG | CH₂ | 3.83 | 3.68 |
| PLA | CH | 5.29 | 4.07 |
| | CH₃ | 1.59 | 1.29 |
| Pglu | CH | 5.88 | 4.28 |
| | (CH—)CH₂ₐ | 2.29 | 1.89 |
| | (CH—)CH_{2b} | 2.59 | 2.00 |
| | (CO2H—)CH₂ | 2.78 | 2.23 |

Experiment 2.8 Triblock Copolymer Poly(glutamic acid-b-lactide-b-ethylene glycol)

| Rgt | m (g) | V (mL) |
|---|---|---|
| PEG-PLA | 0.7066 | |
| PBnGlu | 0.6381 | |
| DCC | 0.0497 | |
| N- | 0.0207 | |
| hydroxysuccinimide | | |
| sodium | | |
| N-methyl | | 13.8 |
| pyrrolidinone | | |

PolyBenzylglutamic acid (0.6381 g, MW=3870 g/mol, prepared according to the procedure described in by H. Kricheldorf, α-aminoacid-N-carboxy anhydride and related heterocycles, Springer-Verlag, 1987 page 88), and the polymer described in experiment 1.8 were suspended in N-methylpyrrolidinone (NMP, 10.7 ml). 20.7 mg of N-hydroxysuccinimide sodium and 49.7 mg of dicyclohexyl carbodiimide (DCC) were dissolved in NMP, in another round bottom flask. Then the two liquids were mixed together, and stirred at 55° C. for 2 hours. The solution was poured in 70 ml of ultrapure water. The solid was collected by filtration on a fritted glass. The solid was washed by trituration with 70 ml of water, then with 70 mL of methanol and finally with 70 mL of ether. The white solid obtained was dried under vacuum.

Then the dried solid was dissolved in 6 mL of Trifluoroacetic acid. The solution was cooled at 10° C. 3.85 mL of methane sulfonic acid and 0.95 mL of anisole were added. After 3 h, under magnetic stirring and at 10° C., 54 mL of cold ether were added and the solid was collected by filtration on a fritted glass. The solid was washed by trituration with 70 mL of ether. The white solid obtained was dried under vacuum.

Analysis:

¹H NMR (400 MHz)

| | | Trifluoroacetic | D2O (δ, |
|---|---|---|---|
| solvent signal | | acid (δ, ppm) | ppm) |
| PEG | CH₂ | 3.55 | 3.41 |
| PLA | CH | 5.72 | 3.82 |
| | CH₃ | 2.01 | 1.04 |
| Pglu | CH | 5.18 | 4.01 |
| | (CH—)CH₂ₐ | 2.51 | 1.63 |
| | (CH—)CH_{2b} | 2.67 | 1.74 |
| | (CO2H—)CH₂ | 2.99 | 1.96 |

Transmission Electron Micrograph (TEM)

Suspensions were prepared by suspending the polymer prepared in Experiment number 2.8 in basic water (final pH between 7 and 8) at a concentration in weight of 0.01% in weight. After 20 minutes of sonication, a drop of the suspension was deposited on a gold grid (Formvar carbon support film on specimen grid, Mesh 400). The electron microscope used was a JEOL 100S TEM with an accelerating voltage of 80 KV. A 2 s exposure time was used to take cliches of the polymer, showing the presence of vesicles (number average diameter=200 nm).

Experiment 2.9 Triblock Copolymer Poly(glutamic acid-b-lactide-b-ethylene glycol)

| Rgt | m (g) | V (mL) |
|---|---|---|
| PEG-PLA | 0.1121 | |
| PBnGlu | 0.2142 | |
| DCC | 0.0170 | |
| N- | 0.0071 | |
| hydroxysuccinimide | | |
| sodium | | |
| N-methyl | | 2.75 |
| pyrrolidinone | | |

PolyBenzylglutamic acid (0.21421 g, MW=6450 g/mol, prepared according to the procedure described in by H. Kricheldorf, α-aminoacid-N-carboxy anhydride and related heterocycles, Springer-Verlag, 1987 page 88), and the polymer described in experiment 1.10 were suspended in N-methyl pyrrolidinone (NMP, 1.7 ml). 7.1 mg of N-hydroxysuccinimide sodium and 17 mg of dicyclohexyl carbodiimide (DCC) were dissolved in NMP, in another round bottom flask. Then the two liquids were mixed together, and stirred at 45° C. for 2 hours. The solution was poured in 15 ml of ultrapure water. The solid was collected by filtration on a fritted glass. The solid was washed by trituration with 40 ml of water and then with 40 mL of methanol. The white solid obtained was dried under vacuum.

Then the dried solid was dissolved in 1 mL of Trifluoroacetic acid. The solution was cooled at 1° C. 0.79 mL of methane sulfonic acid and 0.196 mL of anisole were added. After 3 h, under magnetic stirring and at 1° C., 3 mL of cold ether were added and the solid was collected by filtration on a fritted glass. The solid was washed by trituration with 20 mL of ether. The white solid obtained was dried under vacuum.

Analysis:

¹H NMR (400 MHz)

| | | Trifluoroacetic | D2O (δ, |
|---|---|---|---|
| solvent signal | | acid (δ, ppm) | ppm) |
| PEG | CH₂ | 3.43 | 3.35 |
| PLA | CH | 5.59 | 3.77 |
| | CH₃ | 1.91 | 0.98 |
| Pglu | CH | 5.09 | 3.94 |
| | (CH—)CH₂ₐ | 2.42 | 1.56 |
| | (CH—)CH_{2b} | 2.56 | 1.68 |
| | (CO2H—)CH₂ | 2.90 | 1.89 |

Experiment 2.10 Triblock Copolymer Poly(glutamic acid-b-lactide-b-ethylene glycol)

| Rgt | m (g) | V (mL) |
|---|---|---|
| PEG-PLA | 0.675 | |
| PBnGlu | 2.3963 | |
| DCC | 0.1876 | |
| N- | 0.0781 | |
| hydroxysuccinimide | | |
| sodium | | |
| N-methyl | | 24 |
| pyrrolidinone | | |

PolyBenzylglutamic acid (2.3963 g, MW=6450 g/mol, prepared according to the procedure described in by H. Kricheldorf, α-aminoacid-N-carboxy anhydride and related heterocycles, Springer-Verlag, 1987 page 88), was suspended in N-methyl pyrrolidinone (NMP, 10 ml) and the polymer described in experiment 1.11 was suspended in another round bottom flask in N-methylpyrrolidinone (NMP, 14 ml). 78.1 mg of N-hydroxysuccinimide sodium and 0.1876 g of dicyclohexyl carbodiimide (DCC) were added to the solution containing the PolyBenzylglutamic acid. Then the two liquids were mixed together, and stirred at 45° C. for 2 hours. The solution was poured in 120 ml of ultrapure water. The solid was collected by filtration on a fritted glass. The solid was washed by trituration with 70 ml of water and then with 70 mL of methanol. The white solid obtained was dried under vacuum.

Then the dried solid was dissolved in 11.5 mL of Trifluoroacetic acid. The solution was cooled at 10° C. 6.4 mL of methane sulfonic acid and 1.6 mL of anisole were added. After 3 h, under magnetic stirring and at 10° C., the solution was poured into 130 mL of cold ether and the solid was collected by filtration on a fritted glass. The solid was washed by trituration with 100 mL of ether. The white solid obtained was dried under vacuum.

Analysis:

¹H NMR (400 MHz)

| | | Trifluoroacetic | D2O (δ, |
|---|---|---|---|
| solvent signal | | acid (δ, ppm) | ppm) |
| PEG | CH₂ | 3.32 | 3.46 |
| PLA | CH | 5.51 | 3.87 |
| | CH₃ | 1.82 | 1.08 |
| Pglu | CH | 4.98 | 4.06 |
| | (CH—)CH₂ₐ | 2.32 | 1.68 |
| | (CH—)CH_{2b} | 2.47 | 1.81 |
| | (CO2H—)CH₂ | 2.83 | 2.01 |

Experiment 2.11 Triblock Copolymer Poly(glutamic acid-b-lactide-b-ethylene glycol)

| Rgt | m (g) | V (mL) |
|---|---|---|
| PEG-PLA | 0.2732 | |
| PBnGlu | 1.1305 | |
| DCC | 0.0525 | |
| N- | 0.022 | |
| hydroxysuccinimide | | |
| sodium | | |
| N-methyl | | |
| pyrrolidinone | | |

PolyBenzylglutamic acid (1.1305 g, MW=6450 g/mol, prepared according to the procedure described in by H. Kricheldorf, α-aminoacid-N-carboxy anhydride and related heterocycles, Springer-Verlag, 1987 page 88), was suspended in N-methyl pyrrolidinone (NMP, 5.5 ml) and the polymer described in experiment 1.12 was suspended in another round bottom flask in N-methylpyrrolidinone (NMP, 3.5 ml). 22 mg of N-hydroxysuccinimide sodium and 52.5 mg of dicyclohexyl carbodiimide (DCC) were added to the solution containing the PolyBenzylglutamic acid. Then the two liquids were mixed together, and stirred at 45° C. for 2 hours. The solution was poured in 45 ml of ultrapure water. The solid was collected by filtration on a fritted glass. The solid was washed by trituration with 70 ml of water and then with 70 mL of methanol. The white solid obtained was dried under vacuum.

Then the dried solid was dissolved in 2 mL of Trifluoroacetic acid. The solution was cooled at 10° C. 1 mL of methane sulfonic acid and 0.26 mL of anisole were added. After 3 h, under magnetic stirring and at 10° C., the solution was poured into 3 mL of cold ether and the solid was collected by filtration on a fritted glass. The solid was washed by trituration with 20 mL of ether. The white solid obtained was dried under vacuum.

Analysis:

¹H NMR (400 MHz)

| | | Trifluoroacetic | D2O (δ, |
|---|---|---|---|
| solvent signal | | acid (δ, ppm) | ppm) |
| PEG | CH₂ | 3.50 | 3.51 |
| PLA | CH | 5.68 | 3.91 |
| | CH₃ | 1.98 | 1.13 |
| Pglu | CH | 5.14 | 4.10 |
| | (CH—)CH₂ₐ | 2.48 | 1.71 |
| | (CH—)CH_{2b} | 2.62 | 1.84 |
| | (CO2H—)CH₂ | 2.97 | 2.08 |

Experiment 3.1 Insulin Encapsulation

A solution of human recombinant insulin in Hepes buffer (Aldrich, pH=8.2, [insulin]=10 mg/mL) was diluted ten times with a phosphate buffer (pH=7.4, 300 mOsm). The polymer synthesized in the experiment 2.2 was dissolved in this solution, so that the polymer concentration was 8.2 mg/mL. The pH was adjusted to a value between 7 and 8 using a solution of sodium hydroxide (10 mol/l). The suspension was sonicated for 20 minutes in a sonicating bath at room temperature. After incubation for 3 h, the amount of residual insulin in the suspension was then analyzed in a high pressure liquid chromatography (HPLC) Agilent Hewlet Packard series 1100 HPLC equipped with a ZORBAX 300 SB-C8 5 um column. The eluent was a combination of water/trifluoacetic acid (TFA) (2 mL of TFA in 1 L of ultra pure water) and acetonitrile/TFA (1 mL of TFA in 1 L of acetoniltrile). The elution consisted of a solvent gradient from 80% water/TFA (20% acetonitrile/TFA) to 50% water/TFA (50% acetonitrile/TFA) spread over 15 minutes. The flow rate was 1 mL/min and the elution was done at 30° C. The peak of insulin was monitored at 9.6 min, which corresponds to the peak of a non-encapsulated insulin by comparison to a pure insulin solution. Its area gave access to the amount of insulin which is not encapsulated. The proportion of encapsulated insulin, 21%, was obtained from the mass balance between encapsulated and non-encapsulated insulin.

Experiment 3.2 Insulin Encapsulation

The polymer synthesized in the experiment 2.2 was dissolved in a solution of human recombinant insulin in Hepes buffer (Aldrich, pH=8.2, [insulin]=1.54 mg/mL) so that the polymer concentration was 18.3 mg/mL. The pH was adjusted to a value of 7.5+/−0.5 using a solution of sodium hydroxide 6.5 mol/l. The solution was sonicated for 5 min in a sonicating bath. After incubation for 3 h, the solution was then analyzed in a high pressure liquid chromatography (HPLC) Agilent Hewlet Packard series 1100 HPLC equipped with a ZORBAX 300 SB-C8 5 um column. The eluent was a combination of water/trifluoacetic acid (TFA) (2 mL of TFA in 1 L of ultra pure water) and acetonitrile/TFA (1 mL of TFA in 1 L of acetoniltrile). The elution consisted of a solvent gradient from 80% water/TFA (20% acetonitrile/TFA) to 50% water/TFA (50% acetonitrile/TFA) spread over 15 minutes. The flow rate was 1 mL/min and the elution was done at 30° C. The peak of insulin was monitored at 9.6 min, which corresponds to the peak of a non-encapsulated insulin by comparison to a pure insulin solution. Its area gave access to the amount of insulin which is not encapsulated. The proportion of encapsulated insulin, 23%, was obtained from the mass balance between encapsulated and non-encapsulated insulin.

Experiment 4: Preparation of a Gastroresistant Formulation

Human recombinant insulin (Aldrich, [insulin]=10 mg/ml, in 25 mM Hepes, pH=8.2, sterile-filtered) was diluted 3.4 times with a HEPES buffer (pH=8.2, 25 millimolar of HEPES in water). A suspension was prepared by mixing this insulin solution to the triblock copolymer prepared in experiment 2.1 so that the polymer concentration reaches 15 g/L. The suspension was sonicated for 10 minutes in a Branson 2210 ultrasonic cleaner at room temperature. Eudragit L100 (643 mg) was dissolved in a solution of ethanol (2.15 mL)/acetone (4.3 mL) and 6.1 ml of the suspension of vesicles was poured in the ethanolic solution under magnetic stirring. Span 40 (0.904 g) and Antifoam A (91.7 mg) were then added and the stirring was continued for 30 minutes. Then the suspension was poured in 90.51 g of liquid paraffin (Amojell™ snow white) and homogenized three times during 20 seconds, leaving one minute between each homogenization. The homogenizer was an IKA Labortechnik Ultraturrax™ UT-25-basic instrument equipped with a medium head and operated at 11000 rpm. The system was heated at 40° C. and stirred under magnetic stirring. After three hours, the paraffin was dissolved in 300 mL of hexane. The gastroresistant capsules were filtered over a buchner funnel (medium pore size), washed with hexane (2*100 mL) and then dried under vacuum for 12 hours at room temperature.

Experiment 5: Oral Delivery of Insulin Encapsulated in Polymer 2

The experiment was done with female Sprague-Dawley rats (weight between 180 and 200 g). The rats were delivered with one of their jugular vein cathetered (Charles River company). The rats were fasted for 12 hours prior the experiment. The rats were separated in four groups:

| Group | type of delivery | # rat |
|---|---|---|
| 1 | Subcutaneous injection | 1 to 4 |
| 2 | gavage with insulin solution | 5 to 8 |
| 3 | gavage with insulin encapsulated in polymer | 9 to 11 |
| | 2.1 | |
| 4 | gavage with gastroresistant formulation | 12 to 15 |
| | containing polymer 2.1 | |

Blood samples (200 microliters) were withdrawn several times over a period of 7 hours. Insulin was fed (or injected) immediately after collection of the second blood sample at time 30 minutes. For each sample, the glucose level was measured with a glucometer (Freestyle, Therasense). The blood was poured in EDTA coated tubes (Microvefte 200 um, Sarstedt Inc.) and was centrifuged at 3000 rpm during 15 min. The plasma was isolated and analyzed using an insulin ELISA kit (Human Insulin Elisa Kit, #EZHI-14K, Linco Research, Inc.).

For the each rats of group 1, 100 microliters of a solution of human recombinant insulin ([insulin]=0.0150 mg/mL) were injected via the catheter. This solution of human recombinant insulin obtained from a commercially available solution of human recombinant insulin ([insulin]=10 mg/mL, 25 mM Hepes, pH=8.2, sterile-filtered, Sigma) which was diluted 666 times with a HEPES buffer (pH=8.2, 25 millimolar of HEPES in water). For the rats of the second group, 500 microliters of a solution of human recombinant insulin ([insulin]=1.54 mg/mL) were fed by oral gavage. This solution of human recombinant insulin obtained from a commercially available solution of human recombinant insulin ([insulin]=10 mg/mL, 25 mM Hepes, pH=8.2, sterile-filtered, Sigma) which was diluted 6.5 times with a HEPES buffer (pH=8.2, 25 millimolar of HEPES in water). For the rats of the third group were fed by oral gavage with 500 microliters of a suspension containing insulin and the polymer. This suspension was prepared by mixing the insulin solution used for the rats of group 2 to polymer 2.1 (polymer concentration=15 g/L). The suspension was sonicated for 10 minutes in a Branson 2210 ultrasonic cleaner at room temperature prior to gavage. Each rat of group 4 was fed with one gastroresistant capsules prepared in experiment 4 and weighing 10 mg+/−2 mg. Each capsule contains nominally 4 units of insulin.

Experiment 6 This Experiment Shows that the A Block (Polyglutamic Acid) is Enzymatically Degraded

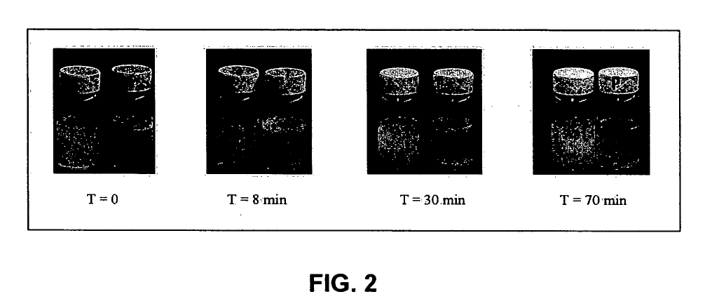

In a first experiment, potassium phosphate tribasic (0.0584 g, 0.2724 10⁻³ mol) and phosphoric acid (0.0213 g, 0.2196 10⁻³ mol) were dissolved in 12 mL of ultra pure water. Then, the triblock copolymer obtained in experiment 2.1 was suspended in this aqueous solution and sonicated for 10 minutes in a sonicating bath. 190 mg of an enzyme (protease, Type I, crude from bovine pancreas, activity 7 units per mg) were added and left in a water bath at 37° C. Pictures were taken at time 0, 8, 30 and 70 minutes (FIG. 2), showing that the vesicles were rapidly degraded by the enzyme. After 30 minutes, only the enzyme was left in solution and the degraded vesicles were precipitated at the bottom of the container.

In a separate experiment, polyglutamic acid (58 mg, MW=12,900 g/mol) was dissolved in a buffer prepared by adding potassium phosphate tribasic (0.0584 g, 0.2724 10⁻³ mol) and phosphoric acid (0.0213 g, 0.2196 10⁻³ mol) in 12 ml of water. The polymer was analyzed by gel permeation chromatography (Agilent 1100) equipped with a dual pump, a degaser, an oven, an autosampler, a UV detector tuned at 254 nm, a column TSK Gel G4000 PWXL and another column TSK Gel G5000 PWXL. The retention time of the polymer was 16.5 min. Then, 25 mg of an enzyme (protease, Type I, crude from bovine pancreas, activity 7 units per mg) was added to the polymeric solution, which was analyzed at regular time intervals by GPC. After 40 minutes, the retention time was 22.3 min. This indicates that polyglutamic acid is rapidly degraded by this enzyme.

In a separate experiment, polyethylene glycol (73 mg, Mₙ=2,000 g/mol) was dissolved in a buffer prepared by adding potassium phosphate tribasic (0.0584 g, 0.2724 10⁻³ mol) and phosphoric acid (0.0213 g, 0.2196 10^{31 3} mol) in 12 ml of water. The polymer was analyzed by gel permeation chromatography (Agilent 1100) equipped with a dual pump, a degaser, an oven, an autosampler, a UV detector tuned at 254 nm, a column TSK Gel G4000 PWXL and another column TSK Gel G5000 PWXL. The retention time of the polymer was 23.5 minutes. Then, 11.5 mg of an enzyme (protease, Type I, crude from bovine pancreas, activity 7 units per mg) was added to the polymeric solution, which was analyzed at regular time intervals by GPC. After 16 hours at 37° C., the retention time was still 23.5 minutes, and the chromatogram of the polymer was unchanged. This indicates that polyethylene glycol is not degraded by this enzyme.

1. A composition comprising a non-phospholipid containing amphiphilic triblock ABC copolymer wherein the A block is characterized as biocompatible, hydrophilic, and enzymatically degradable, wherein the B block is characterized as biocompatible, biodegradable and hydrophobic, and wherein the C block is characterized as biocompatible and hydrophilic, and further wherein the ABC triblock copolymer is characterized by the ability to self assemble into an aqueous vesicle.

2. The composition of claim 1 wherein each of the A, B, and C blocks is characterized by a number average molecular weight of at least 500 g/mol.

3. The composition of claim 1 wherein the aqueous vesicle has an average diameter of about 5 nm to about 10,000 nm.

4. The composition of claim 3 wherein the aqueous vesicle has an average diameter of about 50 nm to about 200 nm.

5. The composition of claim 1 wherein the A block comprises polyglutamic acid.

6. The composition of claim 1 wherein the B block comprises polylactide.

7. The composition of claim 1 wherein the C block comprises polyethylene glycol.

8. The composition of claim 1 wherein the triblock ABC copolymer comprises Poly(glutamic acid-b-lactide-b-ethylene glycol).

9. The composition of claim 1 further comprising an agent characterized by the ability to be encapsulated in the aqueous vesicle.

10. The composition of claim 9 wherein the agent is selected from the group consisting of therapeutic, prophylactic, and diagnostic agents.

11. The composition of claim 9 wherein the agent is selected from the group consisting of a plasmid, oligonucleotide, DNA molecule, RNA molecule, protein, polypeptide, and peptide.

12. An aqueous vesicle comprising a non-phospholipid containing amphiphilic triblock ABC copolymer wherein the A block is characterized as biocompatible, hydrophilic, and enzymatically degradable, wherein the B block is characterized as biocompatible, biodegradable and hydrophobic, and wherein the C block is characterized as biocompatible and hydrophilic, and further wherein the ABC triblock copolymer is characterized by the ability to self assemble into the aqueous vesicle, the vesicle further comprising an agent encapsulated in the aqueous vesicle.

13. The aqueous vesicle of claim 12 wherein each of the A, B, and C blocks is characterized by a number average molecular weight of at least 500 g/mol.

14. The aqueous vesicle of claim 12 wherein the aqueous vesicle has an average diameter of about 5 nm to about 10,000 nm.

15. The aqueous vesicle of claim 14 having an average diameter of about 50 nm to about 200 nm.

16. The aqueous vesicle of claim 12 wherein the A block comprises polyglutamic acid.

17. The aqueous vesicle of claim 12 wherein the B block comprises polylactide.

18. The aqueous vesicle of claim 12 wherein the C block comprises polyethylene glycol.

19. The aqueous vesicle of claim 12 wherein the triblock ABC copolymer comprises Poly(glutamic acid-b-lactide-b-ethylene glycol).

20. The aqueous vesicle of claim 12 wherein the agent is selected from the group consisting of therapeutic, prophylactic, and diagnostic agents.

21. The aqueous vesicle of claim 12 wherein the agent is selected from the group consisting of a plasmid, oligonucleotide, DNA molecule, RNA molecule, protein, polypeptide, and peptide.

22. A method for making a composition for delivery of an agent, the method comprising:
a. providing a non-phospholipid containing amphiphilic triblock ABC copolymer wherein the A block is characterized as biocompatible, hydrophilic, and enzymatically degradable, wherein the B block is characterized as biocompatible, biodegradable and hydrophobic, and wherein the C block is characterized as biocompatible and hydrophilic, and further wherein the ABC triblock copolymer is characterized by the ability to self assemble into the aqueous vesicle; and
b. contacting the non-phospholipid containing amphiphilic triblock ABC copolymer of step a) with an aqueous solution containing the agent to be delivered, effective to form an aqueous vesicle comprising the agent encapsulated in the vesicle comprising the non-phospholipid containing amphiphilic triblock ABC copolymer, thereby forming a composition for the delivery of the agent.

23. The method of claim 22 wherein the aqueous solution is pure or buffered water.

24. The method of claim 22 wherein each of the A, B, and C blocks is characterized by a number average molecular weight of at least 500 g/mol.

25. The method of claim 22 wherein the aqueous vesicle has an average diameter of about 5 nm to about 10,000 nm.

26. The method of claim 25 wherein the aqueous vesicle has an average diameter of about 50 nm to about 200 nm.

27. The method of claim 22 wherein the A block comprises polyglutamic acid

28. The method of claim 22 wherein the B block comprises polylactide.

29. The method of claim 22 wherein the C block comprises polyethylene glycol.

30. The method of claim 22 wherein the triblock ABC copolymer comprises Poly(glutamic acid-b-lactide-b-ethylene glycol)).

31. The method of claim 22 wherein the agent is selected from the group consisting of therapeutic, prophylactic, and diagnostic agents.

32. The method of claim 22 wherein the agent is selected from the group consisting of a plasmid, oligonucleotide, DNA molecule, RNA molecule, protein, polypeptide, and peptide.

33. A method for administering an agent to an animal, the method comprising:
a. providing a composition comprising
i. a non-phospholipid containing amphiphilic triblock ABC copolymer wherein the A block is characterized as biocompatible, hydrophilic, and enzymatically degradable, wherein the B block is characterized as biocompatible, biodegradable and hydrophobic, and wherein the C block is characterized as biocompatible and hydrophilic, and further wherein the ABC triblock copolymer is characterized by the ability to self assemble into an aqueous vesicle; and
ii. an agent to be delivered to an animal, the agent being characterized by the ability to be encapsulated in the aqueous vesicle of i); and
b. administering the composition of step a) to an animal to which administration of the agent is desired.

34. The method of claim 33 wherein the agent is encapsulated in the self-assembled vesicle prior to step b).

35. The method of claim 33 wherein the agent is encapsulated in the self-assembled vesicle subsequent to step b).

36. The method of claim 33 wherein each of the A, B, and C blocks is characterized by a number average molecular weight of at least 500 g/mol.

37. The method of claim 33 wherein the aqueous vesicle has an average diameter of about 5 nm to about 10,000 nm.

38. The method of claim 37 wherein the aqueous vesicle has an average diameter of about 50 nm to about 200 nm.

39. The method of claim 33 wherein the A block comprises polyglutamic acid

40. The method of claim 33 wherein the B block comprises polylactide.

41. The method of claim 33 wherein the C block comprises polyethylene glycol.

42. The method of claim 33 wherein the triblock ABC copolymer comprises Poly(glutamic acid-b-lactide-b-ethylene glycol).

43. The method of claim 33 wherein the agent is selected from the group consisting of therapeutic, prophylactic, and diagnostic agents.

44. The method of claim 33 wherein the agent is selected from the group consisting of a plasmid, oligonucleotide, DNA molecule, RNA molecule, protein, polypeptide, and peptide.

45. The method of claim 33 wherein the composition is administered orally.

46. The method of claim 33 wherein the animal is a human.

47. The method of claim 33 wherein the composition is characterized by the ability to cross the blood-brain barrier.
